(19) United States
(12) Patent Application Publication
(10) Pub. No.: US 2019/0070313 A1
(43) Pub. Date: Mar. 7, 2019

(54) TRI-MODAL NUCLEIC ACID DELIVERY SYSTEMS

(71) Applicant: Amirreza RAFIEE, Kitchener (CA)

(72) Inventor: Amirreza RAFIEE, Kitchener (CA)

(21) Appl. No.: 15/696,945

(22) Filed: Sep. 6, 2017

(51) Int. Cl. A61K 48/00; A61K 47/42; A61K 47/12; A61K 47/18; A61K 47/22; C12N 15/90

(52) U.S. Cl. CPC A61K 48/0033; A61K 47/42; C12N 15/90; A61K 47/186; A61K 47/22; A61K 47/12

(57) ABSTRACT

Provided herein are nucleic acid delivery compositions, surfactants, kits comprising said materials, methods and uses thereof, and methods for the preparation thereof. In particular, nucleic acid delivery compositions described herein may include tri-modal nucleic acid delivery compositions which may comprise at least one peptide enhancer, at least one surfactant, and at least one helper lipid. By way of example, certain of the tri-modal nucleic acid delivery compositions described herein include a peptide enhancer; a surfactant which is a functionalized cationic gemini surfactant; and a helper lipid which is a neutral lipid such as DOPE.

### FIELD OF INVENTION

The present invention relates generally to the delivery of nucleic acids into cells. More specifically, the present invention relates to nucleic acid delivery systems, uses thereof, and methods for the preparation thereof.

### SEQUENCE LISTING

The instant application contains a Sequence Listing which has been submitted electronically in ASCII format and is hereby incorporated by reference in its entirety. Said ASCII copy, created on Sep. 6, 2017, is named 938194 ST25.txt and 3,143 bytes in size.

### BACKGROUND

Delivery of therapeutic genes or other nucleic acids to diseased tissue is challenging and highly sought after in the field of therapeutic research. Cellular uptake and effective endosomal release are the two important components for in vivo application of nucleic acid-based therapies. Non-specific cellular uptake has been attempted by incorporating various alkyl chain lengths into delivery systems, using hydrophobic amino acids, or using cell penetrating peptides to enhance the penetration of nanocarriers across cellular membranes, for example. Peptide ligands such as transferrin, epidermal growth factor, and cell adhesion molecules have been grafted to various delivery systems to target cellular uptake in a site-specific manner [1, 2].

The quaternizing amine group has frequently been used for increasing the cationic charge density for a given vector, and is typically reported to improve transfection efficiency. Promoting endosomal release has been investigated by incorporating various macromolecules bearing unprotonated amine groups with low pKa values to stage endosomal escape due to a so-called “proton sponge” effect [2, 3]. When complexed with DNA and incorporated into the cell, these compounds influx counterions into the engulfed endosomal vesicle, inducing endosomal swelling and lysis, releasing the DNA into the cytoplasm. Polyethylenimine (PEI), histidine or imidazole containing polymers, peptides, and lipids are a few examples of such systems [2, 4, 5].

While the increasing charge density of delivery systems may be effective in enhancing cellular uptake and possibly endosomal rupture, cellular toxicity is another challenge when developing a gene delivery system. Histidine or guanidine functional groups have been shown to lower cellular toxicity due to better distributing of positive charges. The guanidine head group of arginine has also been considered to more effectively improve internalization by forming hydrogen bonds with the negatively charged phosphate and sulfates of cell surface membranes as compared to lysine with the same positive charges [6]. Cysteine residues containing thiol groups have been used to improve colloidal stabilization and transfection efficiency through reducible interpeptide disulfide bonds, therefore forming cross-linked complexes with DNA [7]. However, many studies fail to offer a critical view in distinguishing between the transfection efficiency resulting from the cellular uptake of DNA, and the transfection efficacy associated with successful endosomal escape and gene expression level (in examples where a gene is being delivered). This, therefore, has often previously resulted in inconclusive analysis of nanoparticle transfection profiles.

Alternative, additional, and/or improved nucleic acid delivery compositions and/or methods are desirable.

### SUMMARY OF INVENTION

In one embodiment, there is provided herein a tri-modal nucleic acid delivery composition comprising:
at least one peptide enhancer;
at least one surfactant; and
at least one helper lipid.

In another embodiment of the tri-modal nucleic acid delivery composition above, the peptide enhancer may be zwitterionic, cationic, and/or may comprise at least one histidine, lysine, or arginine residue.

In yet another embodiment of the tri-modal nucleic acid delivery composition or compositions above, the peptide enhancer may comprise an RGD sequence motif.

In still another embodiment of the tri-modal nucleic acid delivery composition or compositions above, the peptide enhancer may comprise an amino acid sequence of P_{A} (GRGDSPG; SEQ ID NO: 1), P_{B} (H(R)₃H(R)₃HG; SEQ ID NO: 2), P_{C} (GRGDSPGH(R)₃H(R)₃HG; SEQ ID NO: 3), P_{D} ((H)₅; SEQ ID NO: 4), P_{E} (GRGDSPG(H)₅; SEQ ID NO: 5), P_{F} ((H)₂R(H)₇R(H)₃G; SEQ ID NO: 6), P_{G} (GRGDSPG(H)₂R(H)₇R(H)₃G; SEQ ID NO: 7), or GRGDSP (SEQ ID NO: 16).

In yet another embodiment of the tri-modal nucleic acid delivery composition or compositions above, the surfactant may comprise a fusogenic surfactant.

In another embodiment of the tri-modal nucleic acid delivery composition or compositions above, the surfactant may comprise a cationic gemini surfactant

In still another embodiment of the tri-modal nucleic acid delivery composition or compositions above, the cationic gemini surfactant may comprise two monomeric surfactants linked by a spacer group.

In yet another embodiment of the tri-modal nucleic acid delivery composition or compositions above, the gemini surfactant may have the formula m-s-m, where m represents the number of alkyl tail carbon atoms of each monomeric surfactant, and s represents the number of atoms in the spacer group.

In another embodiment of the tri-modal nucleic acid delivery composition or compositions above, the surfactant may be functionalized with a functional moiety.

In yet another embodiment of the tri-modal nucleic acid delivery composition or compositions above, the surfactant may be functionalized with the functional moiety by covalent attachment, optionally though a linker.

In still another embodiment of the tri-modal nucleic acid delivery composition or compositions above, the surfactant may be a cationic gemini surfactant, the surfactant may be functionalized with the functional moiety by covalent attachment to a nitrogen atom in the surfactant optionally through a linker, and the nitrogen atom in the surfactant may be a nitrogen atom of a spacer group of the cationic gemini surfactant.

In another embodiment of the tri-modal nucleic acid delivery composition or compositions above, the surfactant may be an m-7NH-m cationic gemini surfactant or a derivative thereof.

In yet another embodiment of the tri-modal nucleic acid delivery composition or compositions above, each m may be independently an integer ≥12 and ≤18, s is ≥3 and ≤7, or both.

In another embodiment of the tri-modal nucleic acid delivery composition or compositions above, the surfactant may be functionalized with a functional moiety which comprises an imidazole-containing functional group, a thiol-containing functional group, a linear RGD-containing peptide functional group, a polyhistidine-containing peptide functional group, a bifunctional RGD-polyhistidine-containing peptide functional group, a zwitterionic and/or cationic arginine-rich peptide functional group, or any combination thereof.

In yet another embodiment of the tri-modal nucleic acid delivery composition or compositions above, the functional moiety may comprise:

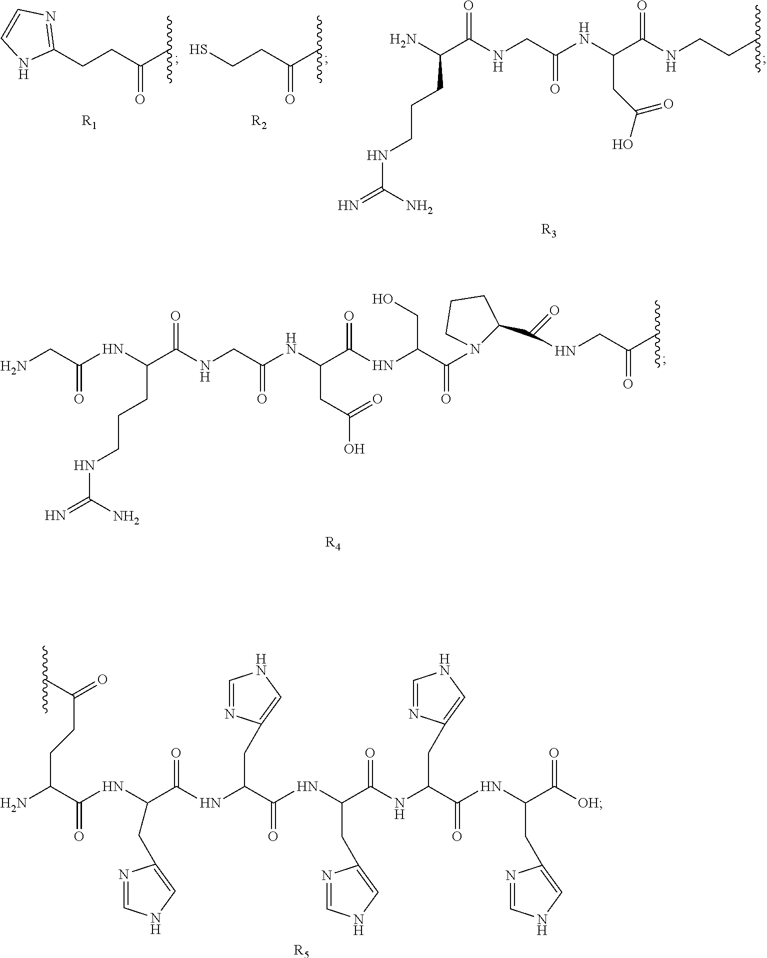

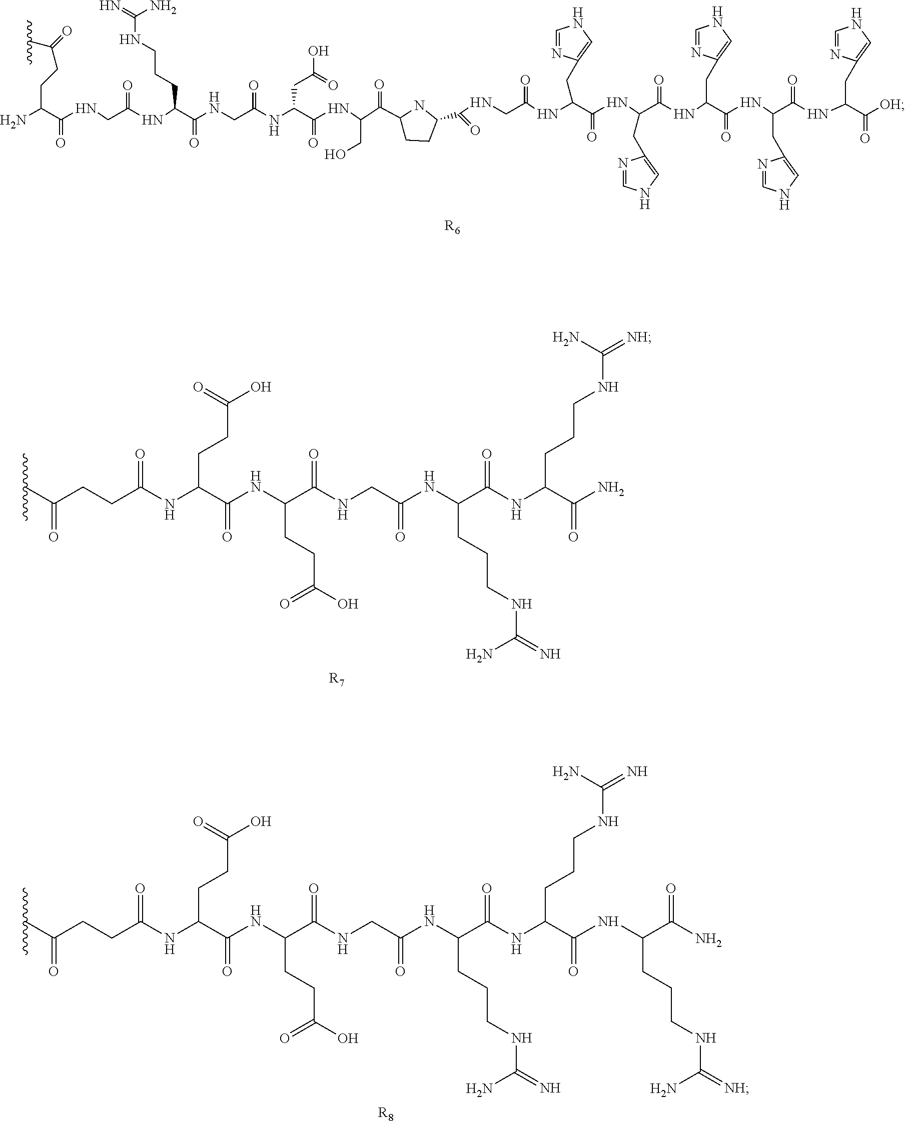

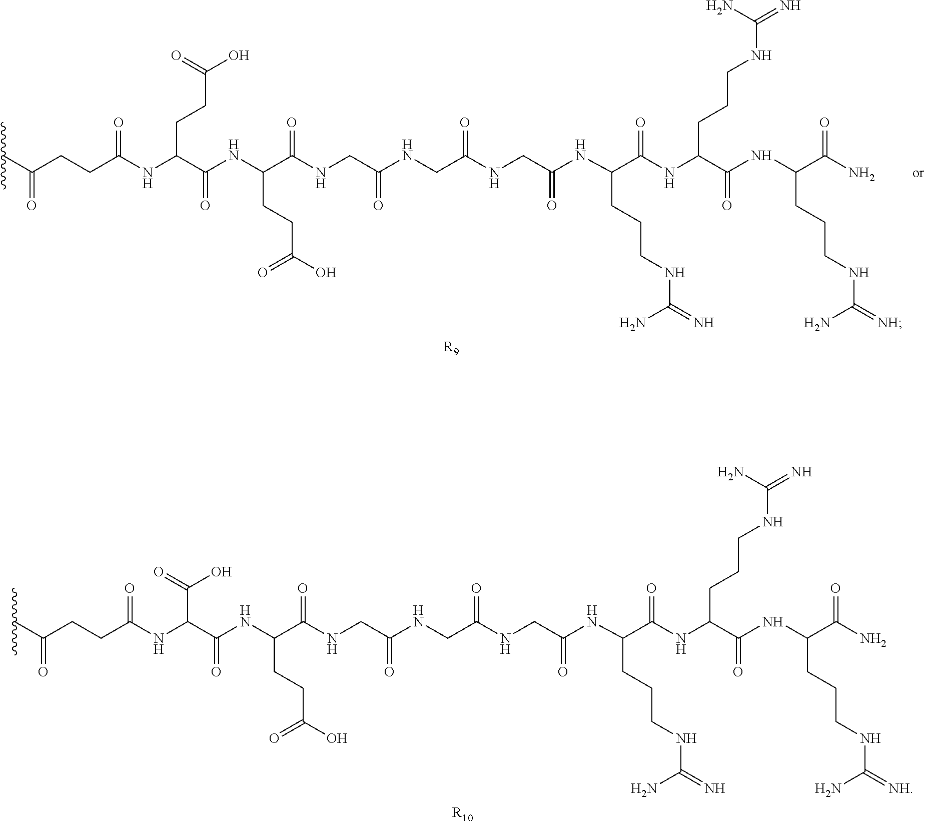

In yet another embodiment of the tri-modal nucleic acid delivery composition or compositions above, the helper lipid may comprise a neutral helper lipid.

In still another embodiment of the tri-modal nucleic acid delivery composition or compositions above, the helper lipid may comprise DOPE (1,2-dioleyl-sn-glycero-3-phosphoethanolamine), DPPC (1,2-dipalmitoyl-sn-glycero-3-phosphocholine), a derivative thereof, or any combination thereof.

In another embodiment of the tri-modal nucleic acid delivery composition or compositions above, the composition may further comprise a nucleic acid for delivery to a cell.

In yet another embodiment of the tri-modal nucleic acid delivery composition or compositions above, the nucleic acid may comprise a plasmid, expression vector, therapeutic nucleic acid, or another nucleic acid molecule.

In yet another embodiment of the tri-modal nucleic acid delivery composition or compositions above, the composition may have a cationic surfactant/nucleic acid charge ratio (ρ) of ρ≤3.

In still another embodiment of the tri-modal nucleic acid delivery composition or compositions above, the composition may have a helper lipid/surfactant molar ratio (r) of r≤10.

In yet another embodiment of the tri-modal nucleic acid delivery composition or compositions above, the composition may have a molar concentration of peptide enhancer (M_{P}) of M_{P}≤1000 μM, a molar concentration of surfactant (M_{G}) of M_{G}≤46 μM, and/or a molar concentration of helper lipid (M_{L}) of M_{L}≤300 μM.

In still another embodiment of the tri-modal nucleic acid delivery composition or compositions above, the composition may have a surface charge (ζ potential) of −60 mV≤ζ≤60 mV.

In yet another embodiment of the tri-modal nucleic acid delivery composition or compositions above, the composition may have a particle size of ≥80 nm and ≤350 nm.

In yet another embodiment, there is provided herein a kit for delivering a nucleic acid to a cell, the kit comprising a tri-modal nucleic acid delivery composition as described hereinabove and, optionally, instructions for formulating the nucleic acid with the tri-modal nucleic acid delivery composition.

In still another embodiment, there is provided herein a method of delivering a nucleic acid to a cell, said method comprising:
generating a delivery vehicle comprising the nucleic acid by formulating the nucleic acid with the tri-modal nucleic acid delivery composition as described hereinabove; and
administering the delivery vehicle to the cell.

In yet another embodiment, there is provided herein a use of the tri-modal nucleic acid delivery composition as described herein above for delivering a nucleic acid to a cell.

In still another embodiment, there is provided herein a method of preparing a nucleic acid for delivery to a cell, said method comprising:
formulating the nucleic acid with the tri-modal nucleic acid delivery composition as described hereinabove.

In still another embodiment, there is provided herein a gemini surfactant comprising two monomeric surfactants linked by a spacer group, the gemini surfactant being covalently functionalized with a functional moiety.

In yet another embodiment of the gemini surfactant above, the functional moiety may comprise an imidazole-containing functional group, a thiol-containing functional group, a linear RGD-containing peptide functional group, a polyhistidine-containing peptide functional group, a bifunctional RGD-polyhistidine-containing peptide functional group, a zwitterionic and/or cationic arginine-rich peptide functional group, or any combination thereof.

In still another embodiment of the gemini surfactant or surfactants above, the gemini surfactant may comprise the structure of formula II:

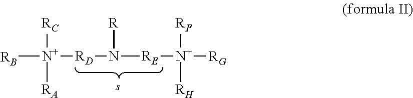

wherein at least one of R_{A}, R_{B}, and R_{C} of a first monomeric surfactant portion may comprise an alkyl-based tail having m₁ carbon atoms, and the remaining of R_{A}, R_{B}, and R_{C} are substituents, such as alkyl (for example, C₁-C₄ alkyl) substituents or an imidazole-based or thiol-based or hydroxyl-based group (for example), which cause the nitrogen to which they are attached to be quaternary;
wherein at least one of R_{F}, R_{G}, and R_{H} of a second monomeric surfactant portion may comprise an alkyl-based tail having m₂ carbon atoms, and the remaining of R_{F}, R_{G}, and R_{H} are substituents, such as alkyl (for example, C₁-C₄ alkyl) substituents or an imidazole-based or thiol-based or hydroxyl-based group (for example), which cause the nitrogen to which they are attached to be quaternary; and
wherein spacer —R_{D}—N(R)—R_{E}— links the first and second monomeric surfactant portions through their respective quaternary nitrogens, R_{D} and R_{E} each represent an alkyl-based group or derivative thereof, R represents the functional moiety and is covalently joined to the nitrogen of the spacer, and s represents the total number of spacer atoms along the shortest linear path running between the quaternary nitrogens of the first and second monomeric surfactant portions.

In yet another embodiment of the gemini surfactant or surfactants above, the gemini surfactant may comprise the structure of formula III:

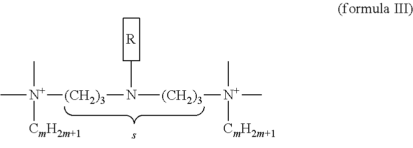

wherein 12≤m≤18, and m may be the same, or different, between the two monomeric surfactant portions;
wherein s is 7; and
wherein R is the functional moiety.

In yet another embodiment of the gemini surfactant above, m may be 12 or 18, and may be the same for both monomeric surfactant portions.

In still another embodiment of the gemini surfactant or surfactants above, the functional moiety may comprise any one of R₁-R₁₀ as defined hereinabove.

In yet another embodiment, there is provided herein a composition comprising the gemini surfactant as defined hereinabove, and at least one of a peptide enhancer, a helper lipid, a nucleic acid, a pharmaceutically acceptable excipient, diluent, or buffer.

In still another embodiment, there is provided herein a kit for delivering a nucleic acid to a cell, the kit comprising the gemini surfactant as defined hereinabove, and, optionally, one or more of a peptide enhancer, a helper lipid, a nucleic acid, or instructions for formulating the nucleic acid with the gemini surfactant.

In another embodiment, there is provided herein a use of the gemini surfactant as defined herein above for delivering a nucleic acid to a cell. In certain embodiments, the gemini surfactant may be for use in combination with at least one peptide enhancer and/or at least one helper lipid.

In yet another embodiment, there is provided herein a method of delivering a nucleic acid to a cell, said method comprising:
formulating the nucleic acid with the gemini surfactant as defined hereinabove; and
administering the formulated nucleic acid to the cell.

In another embodiment of the method above, the formulating step may additionally comprise formulating the nucleic acid with a peptide enhancer and/or a helper lipid.

In another embodiment, there is provided herein a method of preparing a nucleic acid for delivery to a cell, said method comprising:
formulating the nucleic acid with the gemini surfactant as defined hereinabove.

In yet another embodiment of the above method or methods, the formulating step may additionally comprise formulating the nucleic acid with a peptide enhancer and/or a helper lipid.

### BRIEF DESCRIPTION OF DRAWINGS

FIG. 1 shows (A) General structure of m-7NR-m gemini surfactants (m=12, 18; R=R₁-R₁₀), and (B) Chemical structures of functional moieties R₁-R₁₀;

FIG. 2 shows synthetic schemes for R-functionalization of gemini surfactants by Method (A) in solution (for synthesis of G4-G8 m-7NR-m gemini surfactants (m=12, 18; R=R₁-R₄)) or by Method (B) in solid phase (for synthesis of G9-G14 m-7NR-m gemini surfactants (m=18; R=R₅-R₁₄)). m-7NR-m gemini surfactants were synthesized by covalent linking of the imino groups of the m-7NH-m gemini surfactants to free carboxylic groups located either at the C-terminus of the protected R functional motifs (Method (A)) or at the N-terminus of the protected R functional motifs (Method (B)). The cleavage and/or deprotection, and purification steps were accomplished to yield G4-G14 m-7NR-m gemini surfactants;

FIG. 3 shows physicochemical characterizations of gene delivery formulations, determined by dynamic light scattering. (A) Particle size and (B) ζ-potential of Bi-Modal (BM [G M_{G}/L M_{L}]) gene delivery systems formulated using G6 or G7 m-7NR-m gemini surfactants at M_{G}=154 μM, 31 μM and DOPE helper lipids (L) at M_{L}=500 μM, 100 μM. The Optimized BM [G/L] delivery systems were formulated at M_{G}=31 μM and M_{L}=100 μM (i.e., OBM [G 31/L 100]). (C) Particle size and (D) ζ-potential of Uni-Modal (UM [P M_{P}]) delivery systems formulated using zwitterionic peptide enhancers (i.e., =100 P_{A}) at M_{P}=62 μM, 308 μM and cationic peptide enhancers (i.e., P_{B}, P_{C}) at M_{P}=10 μM, 49 μM, 98 μM;

FIG. 4 shows (A) Particle size and (B) ζ-potential of Bi-Modal (BM [P M_{P}/L M_{L}], BM [P M_{P}/G M_{G}]) and Tri-Modal (PDTMG [P M_{P}/G M_{G}/L M_{L}]) delivery formulations, measured by dynamic light scattering;

FIG. 5 shows transfection efficiency, efficacy and cell viability of the BM [G M_{G}/L M_{L}]) delivery systems, investigated by flow cytometry. BM [G M_{G}/L M_{L}]) delivery systems were formulated using m-7NH-m (G2, G3) or m-7NR-m gemini surfactants (G6 or G7) at M_{G}=154 μM (ρ=10), 77 μM (ρ=5), 31 μM (ρ=2) and DOPE helper lipids (L) at M_{L}=500 μM, 300 μM, 100 μM. *M is mock DNA. (A) Transfection efficiency was measured by the percentage of cells transfected with pDNA. (B) Transfection efficacy was measured by the mean florescence intensity of cells expressing GFP. (C) Viability was measured by metabolic activity of the mitochondria;

FIG. 6 shows transfection efficiency, efficacy and cell viability of the Uni-Modal (UM [P_{C} M_{P}]), Bi-Modal (BM [G7 M_{G}/L M_{L}], BM [P_{C} M_{P}/L M_{L}], BM [P_{C} M_{P}/G7 M_{G}]) and Tri-Modal (PDTMG [P_{C}/G7/L]) delivery systems, assessed by flow cytometry. *M is mock DNA. PDTMG-1, PDTMG-2 and PDTMG-3 were formulated according to Table 4. Transfection efficiency was recorded based on the percentage of pDNA-transfected cells (A); transfection efficacy was measured by the MFI of cells expressing GFP protein (B); cell viability was measured by the metabolic activity of cells stained by MitoTracker Deep Red;

FIG. 7 shows PDTMG [P_{C} M_{P}/G7 M_{G}/L M_{L}] delivery systems were optimized by varying ρ values, and molar concentrations of P_{C} cationic peptide enhancers (M_{P}), G7 gemini surfactants (M_{G}) and DOPE helper lipids (M_{L}) to develop a potent delivery system with both high transfection efficiency (A) and efficacy (B);

FIG. 8 shows the impact of non-covalent addition of zwitterionic (i.e. P_{A}) and cationic peptide enhancers (i.e. P_{B-G}) on transfection efficiency (A), efficacy (B) and cell viability (C) of PDTMG [P_{A-G} M_{P}/G7 M_{G}/L M_{L}] delivery systems. PDTMG-1 and PDTMG-2 were formulated according to Table 4;

FIG. 9 shows the impact of thirteen non-functionalized or functionalized gemini surfactants (m-s-m formula; m=12, 18; s=3, 7NH, 7NR) on transfection efficiency (A), efficacy (B) and cell viability (C) of formulated PDTMG-3 [P_{C} 267/G7 17/L 113].

FIG. 10 shows transfection efficiency, efficacy and cell viability of non-functionalized G3 gemini surfactants vs. RGD-functionalized G7 gemini surfactants formulated either in BM [G7 154/L 500], OBM [G7 31/L 100], PDTMG-1 or PDTMG-3. Flow cytometry analysis in 2D dot/density plots of the intensity of green fluorescence (in BL-1 channel) vs. MitoTracker stain signals (in RL-1 channel) were depicted for (A) control groups, (B) BM gene delivery systems and (C) PDTMG delivery systems. Transfection efficiency were recorded by the percentage of pDNA (gWiz™ GFP or *Mock pDNA) positive cells, the transfection efficiency was investigated by the MFI of GFP-expressing cells, and cell viability were reported by the percentage of live cells presenting RL-1 signals. RGD-functionalized G7 gemini surfactants-formulated PDTMG-3 nanoparticles significantly improve cellular uptake of gWiz™ GFP pDNA and enhance the mean fluorescence intensity of cells expressing GFP protein;

FIG. 11 shows a schematic showing the transfection pathways of PDTMG nanoparticles. Schematic depiction of Bi-Modal [G M_{G}/L M_{L}] nanoparticles and PDTMG [P M_{P}/G M_{L}] nanoparticles that can be formulated using peptide enhancers (e.g., P_{C}), fusogenic functionalized gemini surfactants (e.g., G7 and G8) and DOPE helper lipids (L);

FIG. 12 shows (A) the cellular uptake of gWiz™ GFP pDNA, (B) the mean florescence intensity of cells expressing GFP and (C) cell viability of PDTMG-3, PDTMG-Max and commercially available Lipofectamine™3000 reagent. PDTMG-3 and PDTMG-Max were formulated using fusogenic G7 or G8 gemini surfactants and both revealed higher or comparable transfection efficiency and efficacy as compared to Lipofectamine™3000 reagent;

FIG. 13 shows (A) the synthesized R-functionalized G4-G8 gemini surfactants by Method (A). (B) The synthesized R-functionalized G9-G14 gemini surfactants by Method (B);

FIG. 14 shows ESI-MS data to confirm the identity of synthesized G4 (18-7NR₁-18) gemini surfactants;

FIG. 15 shows ESI-MS data to confirm the identity of synthesized G5 (18-7NR₂-18) gemini surfactant;

FIG. 16 shows ESI-MS data to confirm the identity of synthesized G6 (12-7NR₃-12) gemini surfactant;

FIG. 17 shows ESI-MS data to confirm the identity of synthesized G7 (18-7NR₃-18) gemini surfactant;

FIG. 18 shows ESI-MS data to confirm the identity of synthesized G8 (18-7NR₄-18) gemini surfactant;

FIG. 19 shows ESI-MS data to confirm the identity of synthesized G9 (18-7NR₅-18) gemini surfactant;

FIG. 20 shows ESI-MS data to confirm the identity of synthesized G10 (18-7NR₆-18) gemini surfactant;

FIG. 21 shows ESI-MS data to confirm the identity of synthesized G11 (18-7NR₇-18) gemini surfactant;

FIG. 22 shows ESI-MS data to confirm the identity of synthesized G12 (18-7NR₈-18) gemini surfactants;

FIG. 23 shows ESI-MS data to confirm the identity of synthesized G13 (18-7NR₉-18) gemini surfactant; and

FIG. 24 shows ESI-MS data to confirm the identity of synthesized G14 (18-7NR₁₀-18) gemini surfactant.

### DETAILED DESCRIPTION

Described herein are nucleic acid delivery systems, uses thereof, and methods for the preparation thereof. In particular, tri-modal nucleic acid delivery compositions are provided which may comprise at least one peptide enhancer, at least one surfactant, and at least one helper lipid. Surfactants for delivering nucleic acids are also described in detail herein. It will be appreciated that embodiments and examples are provided herein for illustrative purposes intended for those skilled in the art, and are not meant to be limiting in any way.

In certain embodiments, there is provided herein a tri-modal nucleic acid delivery composition comprising:
at least one peptide enhancer;
at least one surfactant; and
at least one helper lipid.

As will be understood, tri-modal nucleic acid delivery compositions may be considered as compositions comprising at least three modalities, such as a peptide enhancer modality, a surfactant modality, and a helper lipid modality, which may function together for cellular delivery. It is contemplated that tri-modal nucleic acid delivery compositions may encompass additional elements, so long as all of a peptide enhancer, a surfactant, and a helper lipid are present. By way of example, tri-modal delivery compositions may, in certain embodiments, additionally comprise a nucleic acid sequence, a peptide sequence, or a therapeutic or targeting moiety, for example.

Nucleic acid delivery compositions described herein may be for use in delivering a nucleic acid cargo to a cell. In certain embodiments, such delivery may be considered as a transection. Nucleic acids may include any suitable nucleic acid for which delivery to a cell may be desired. By way of example, nucleic acids may include plasmids, expression vectors, therapeutic nucleic acids (such as, but not limited to, siRNA, antisense oligonucleotides, miRNA, other small RNAs or DNAs), chemically modified nucleic acids, CRISPR nucleic acids, or any other suitable nucleic acid sequence (DNA, RNA, DNA and RNA, or nucleic acid comprising chemical modifications or fusions) or interest.

While the delivery compositions are described herein primarily in relation to the delivery of nucleic acids, it is contemplated that delivery compositions described herein may be used for delivery of other cargo such as a therapeutic agent or drug which is not a nucleic acid. By way of example, in certain embodiments, it is contemplated that delivery compositions described herein may be for use in delivering nucleic acids in combination with at least one other therapeutic agent or drug (which may be covalently joined to the nucleic acid, complexed with the nucleic acid, or separate from the nucleic acid), or may be for use in delivering at least one therapeutic agent or drug which is not a nucleic acid alone (i.e. without a nucleic acid present).

As will be understood, nucleic acid delivery compositions described herein may include each component (i.e. the peptide enhancer, surfactant, and/or helper lipid) in a separate, unmixed form, or mixed with one or more other components, or with all three components formulated together, for example. The compositions may already be in a form suitable for the delivery of nucleic acids, or may be in a pre-formulation state which can be used to generate a formulation suitable for the delivery of nucleic acids.

In certain embodiments, the peptide enhancer may be any suitable peptide-containing moiety which is able to complex with the nucleic acid, stabilize particles, and/or assist with cellular and/or intracellular delivery thereof. By way of example, in certain embodiments the peptide enhancer may be zwitterionic, cationic, and/or may comprise at least one histidine, lysine, or arginine residue. In certain further embodiments, the peptide enhancer may comprise an RGD amino acid sequence motif. By way of non-limiting example, in certain embodiments, the peptide enhancer may comprise an amino acid sequence of P_{A} (GRGDSPG; SEQ ID NO: 1), P_{B} (H(R)₃H(R)₃HG; SEQ ID NO: 2), P_{C} (GRGDSPGH(R)₃H(R)₃HG; SEQ ID NO: 3), P_{D} ((H)₅; SEQ ID NO: 4), P_{E} (GRGDSPG(H)₅; SEQ ID NO: 5), P_{F} ((H)₂R(H)₇R(H)₃G; SEQ ID NO: 6), P_{G} (GRGDSPG(H)₂R(H)₇R(H)₃G; SEQ ID NO: 7), or GRGDSP (SEQ ID NO: 16).

In certain embodiments, the surfactant may comprise any suitable surfactant which is able to complex or envelop the nucleic acid and assist with the delivery thereof. In certain embodiments, the surfactant may comprise a cationic surfactant. By way of example, in certain embodiments the surfactant may comprise a cationic gemini surfactant. Such cationic gemini surfactants may include those wherein the cationic gemini surfactant comprises two monomeric surfactants linked by a spacer group, and the spacer group may, optionally, be functionalized with a functional moiety.

In certain embodiments, gemini surfactants may include those having the formula:

m-s-m (formula I);
wherein each m independently represents the number of alkyl tail carbon atoms of each respective monomeric surfactant portion, and s represents the number of atoms in the spacer group linking the two surfactant portions.

In certain embodiments, gemini surfactants may include those having higher interfacial activity, self-aggregation property, and/or lower critical micelle concentration (CMC) (in certain further embodiments, about 1 to 2 orders of magnitude lower), as compared to the monomeric surfactants. Examples of gemini surfactants for use in nucleic acid delivery carriers have been described [10, 14, 17, 20].

By way of example, a gemini surfactant of formula II may be considered as an embodiment of a gemini surfactant of formula I as follows:

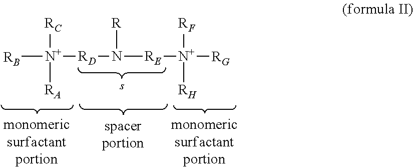

wherein at least one of R_{A}, R_{B}, and R_{C} comprises an alkyl-based tail having m carbon atoms, and the remaining of R_{A}, R_{B}, and R_{C} are substituents, such as alkyl (for example, C₁-C₄ alkyl) substituents or an imidazole-based or thiol-based group, or a hydroxyl-based group (for example), which cause the nitrogen to which they are attached to be quaternary (in certain embodiments, 12≤m≤18);
wherein at least one of R_{F}, R_{G}, and R_{H} comprises an alkyl-based tail having m carbon atoms (which may be the same, or different, from m defined above), and the remaining of R_{F}, R_{G}, and R_{H} are substituents, such as alkyl (for example, C₁-C₄ alkyl) substituents or an imidazole-based or thiol-based group, or a hydroxyl-based group (for example), which cause the nitrogen to which they are attached to be quaternary (in certain embodiments, 12≤m≤18); and
wherein the spacer —R_{D}—N(R)—R_{E}— links the two monomeric surfactant portions through their respective quaternary nitrogens, R_{D} and R_{E} each represent an alkyl-based group or derivative thereof, R represents a functional moiety joined to the nitrogen of the spacer, and s represents the total number of spacer atoms along the shortest linear path running between the quaternary nitrogens. In certain embodiments, 3≤s≤7. As well, in certain embodiments, R_{D} and R_{E} may have the same lengths, or different lengths. In certain embodiments, R_{D} and R_{E} may each comprise 1, 2, 3, 4, or 5 methylene units, for example. In certain embodiments, R functional moieties may include hydrophobic moieties or hydrophilic moieties. In certain embodiments, R functional moieties may comprise a thiol group, imidazole group, or an amino acid residue. In certain embodiments, R functional groups may comprise a histidine, arginine, lysine, and/or glutamic acid residue, or a suitable combination thereof.

By way of further example, a gemini surfactant of formula III may be considered as an embodiment of a gemini surfactant of formulas I and II as follows:

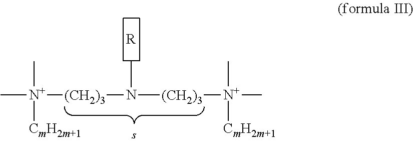

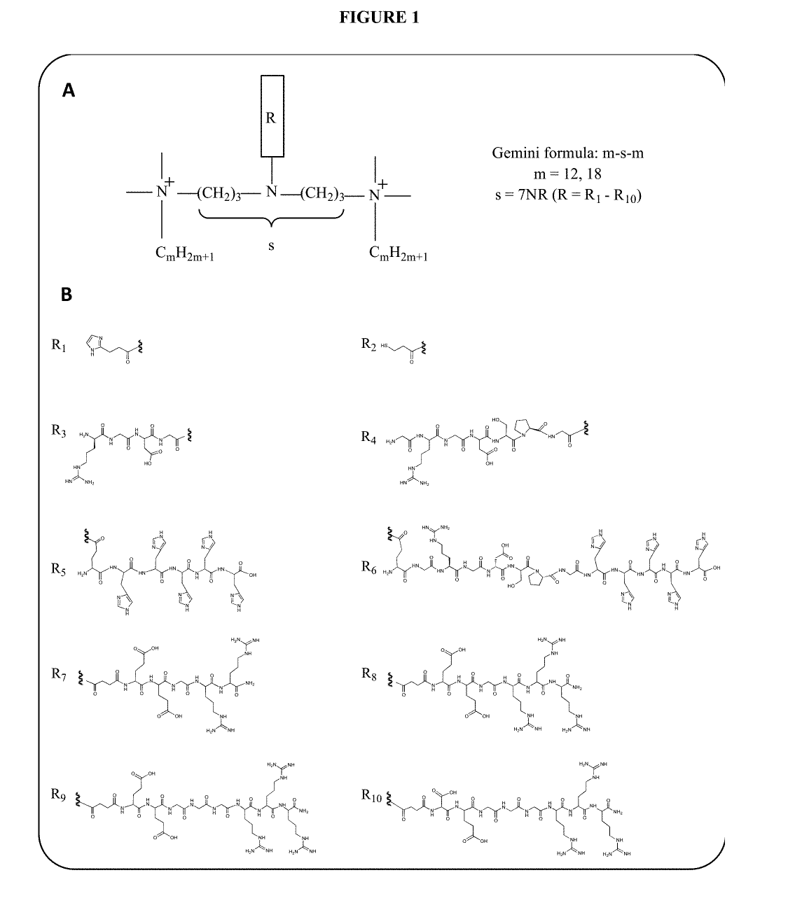

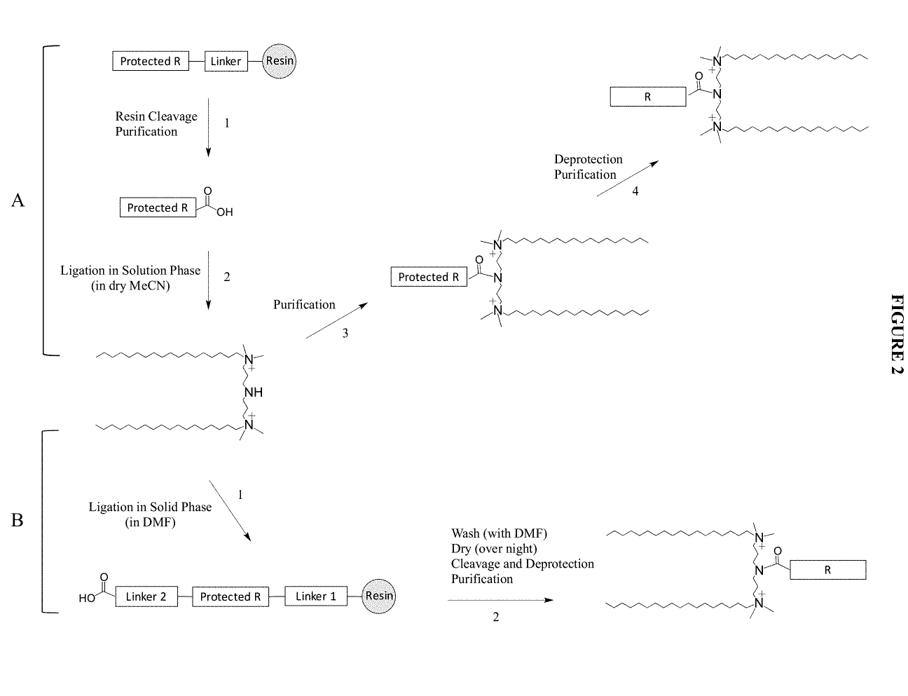

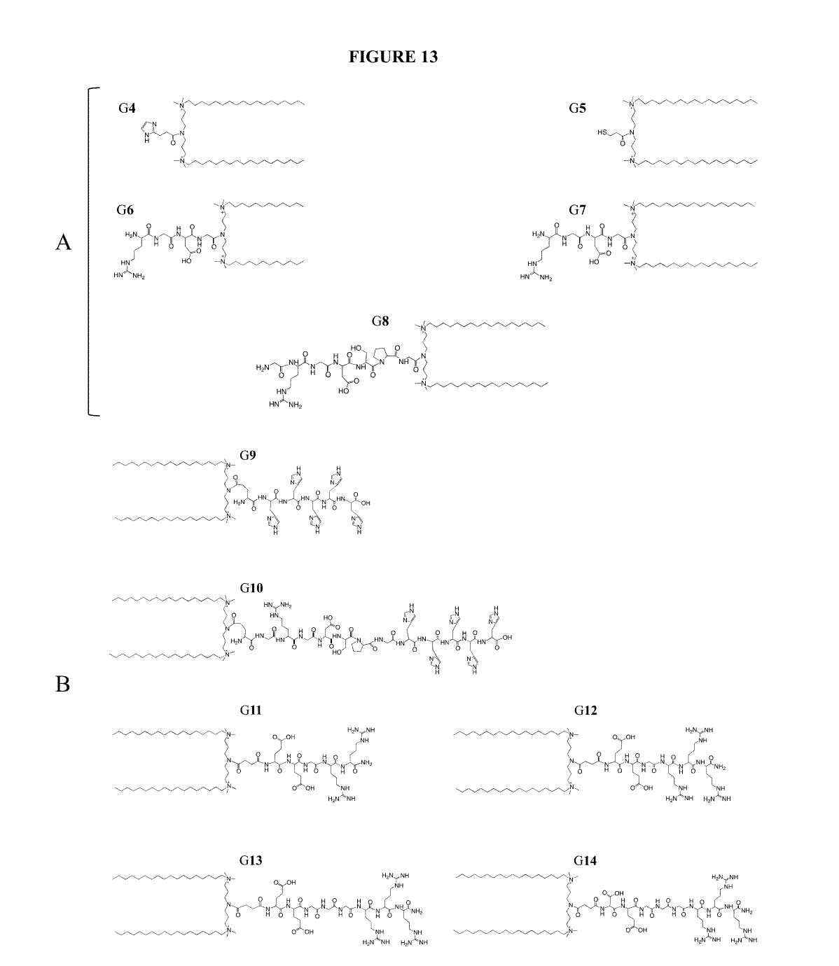

As shown above, formula III is characterized by the structure m-s-m of formulas I and II, wherein two quaternary nitrogen-based surfactants, each having two methyl groups and an alkyl tail on their quaternary nitrogens, are linked together via a —CH₂—CH₂—CH₂—N(R)—CH₂—CH₂—CH₂—spacer (abbreviated 7NR, derived from 7NH, where s is 7) joining the two quaternary nitrogens. In certain embodiments, 12≤m≤18. As will be understood, R represents a functional moiety covalently joined to the nitrogen atom of the spacer. In certain embodiments, R may, for example, be selected from R₁-R₁₀ as shown in FIG. 1. In certain embodiments, cationic gemini surfactants may include those based on N,N-bis(dimethylalkyl)-α,ω-alkanediammonium. By way of non-limiting example, in certain embodiments, cationic gemini surfactants may include G4-G14 as shown in FIG. 13.

Gemini surfactants have been previously described, and in certain embodiments may include those based on or described in [10, 14, 16, 17, 20], for example, which are herein incorporated by reference in their entireties.

Functional moieties as described herein may include any suitable moiety which may be covalently joined to the surfactant (optionally via a linker), and which may assist with cellular uptake and/or cellular targeting and/or endosomal escape of the nucleic acid delivery compositions. In certain embodiments, functional moieties may include any suitable fusogenic peptide or derivative thereof. In certain embodiments, the functional moiety may comprise an RGD amino acid sequence motif. In certain embodiments, functional moieties may include those comprising an imidazole-containing functional group, a thiol-containing functional group, a linear RGD-containing peptide functional group, a polyhistidine-containing peptide functional group, a bifunctional RGD-polyhistidine-containing peptide functional group, a zwitterionic and/or cationic arginine-rich peptide functional group, or any combination thereof. Non-limiting examples of functional moieties may include those shown in FIG. 1 as R₁-R₁₀, or another suitable functional moiety. By way of example, in certain embodiments, R functional moieties may comprise functional peptide moieties designed by alternating amino acid residues with Gly residue to align the side chain of amino acids (i.e., positively charged and/or negatively charged and/or uncharged side chain groups) in order to increase the endosomal destabilizing effect of the delivery system (e.g., XGXGXG, where X represents amino acid residues, and G refer to Gly residues).

In certain embodiments, it is contemplated that cellular delivery may be targeted. By way of example, in certain embodiments targeting moieties such as, but not limited to, transferrin, epidermal growth factor, and/or cell adhesion molecules may be covalently joined to surfactants described herein as an additional element for site-specific targeting (see [1,2]).

In certain embodiments of the above-described gemini surfactants, each m may be independently an integer ≥12 and ≤18 (including any individual integer therebetween), s may be ≥3 and ≤7 (including any individual integer therebetween), or both.

As will be recognized, in certain embodiments, cationic gemini surfactants as described herein may be used for delivering nucleic acids to cells. It is contemplated that in certain embodiments, gemini surfactants as described herein, while being highly amenable for use as part of the tri-modal delivery compositions described herein, may also be used alone, or as part of other nucleic acid delivery systems for achieving cellular uptake. By way of example, in certain embodiments nucleic acid delivery systems may comprise surfactants and peptide enhancers (i.e. in a bimodal delivery system).

As will also be understood, a helper lipid may comprise any suitable lipid or derivative thereof which is able to function along with the surfactant to assist in cellular delivery. In certain embodiments, the helper lipid may comprise a neutral helper lipid. By way of non-limiting example, in certain embodiments the helper lipid may comprise DOPE (1,2-dioleyl-sn-glycero-3-phosphoethanolamine), DPPC (1,2-dipalmitoyl-sn-glycero-3-phosphocholine), suitable derivative(s) thereof, or any combination thereof.

As will be understood, in certain embodiments the tri-modal nucleic acid delivery compositions described herein may further comprise the nucleic acid to be delivered to the cell. By way of example, nucleic acids may include plasmids, expression vectors, therapeutic nucleic acids (such as, but not limited to, plasmid DNA (pDNA), shRNA plasmids, siRNA, antisense oligonucleotides, miRNA, other small RNAs or DNAs), chemically modified nucleic acids, CRISPR nucleic acids, or any other suitable nucleic acid sequence (DNA, RNA, DNA and RNA, or nucleic acid comprising chemical modifications or fusions) or interest.

In certain embodiments, the cells to which the nucleic acid is to be delivered using the nucleic acid delivery compositions may include any suitable cell type such as, but not limited to, fibroblasts, melanoma, epithelial cells, and/or keratinocytes, or other suitable cells, for example.

In certain embodiments, the tri-modal nucleic acid delivery composition described herein (in examples where a gene or pDNA is being delivered) may include those having a cationic gemini surfactant/DNA+/−charge ratio (ρ) of ρ≤3, such as 0.7≤p≤3 (once formulated with the nucleic acid to be delivered). As will be understood, in certain embodiments, adjusting the ρ value may be particular relevant for pDNA complexation, cellular uptake and endosomal escape. The extent of DNA compaction primarily relates to the length of the alkyl tails of gemini surfactants and secondly to the polarity of the head groups. The longer the alkyl tails of gemini surfactants, the tighter the compaction of DNA. In certain embodiments, the ρ value may be optimized according to the alkyl chains of the cationic gemini surfactants to increase the endosomal destabilizing and release of pDNA into the cell cytoplasm. By way of example, the compaction/complexation of pDNA using 18-series dicationic gemini surfactants (m=18) may increase up to ρ≈2, above which the higher compaction/complexation may become detrimental to endosomal release of pDNA in certain examples.

In still further embodiments, the tri-modal nucleic acid delivery compositions described herein (in examples where a gene or pDNA is being delivered) may have a helper lipid/gemini surfactant molar ratio (r) of r≤10, such as 1.5≤r≤10. In certain embodiments, by tuning the r value, physicochemical properties of the delivery systems (i.e., particle stability, size, and/or surface charge potential)) may be improved. This may, for example, further improve the percentage of the cells uptaking pDNA (transfection efficiency) and/or the gene expression level associated with endosomal escape and intracellular delivery of pDNA (transfection efficacy). The r value may be adjusted in correlation with the ρ value to increase the transfection efficiency and efficacy of the tri-modal gene delivery systems in certain embodiments.

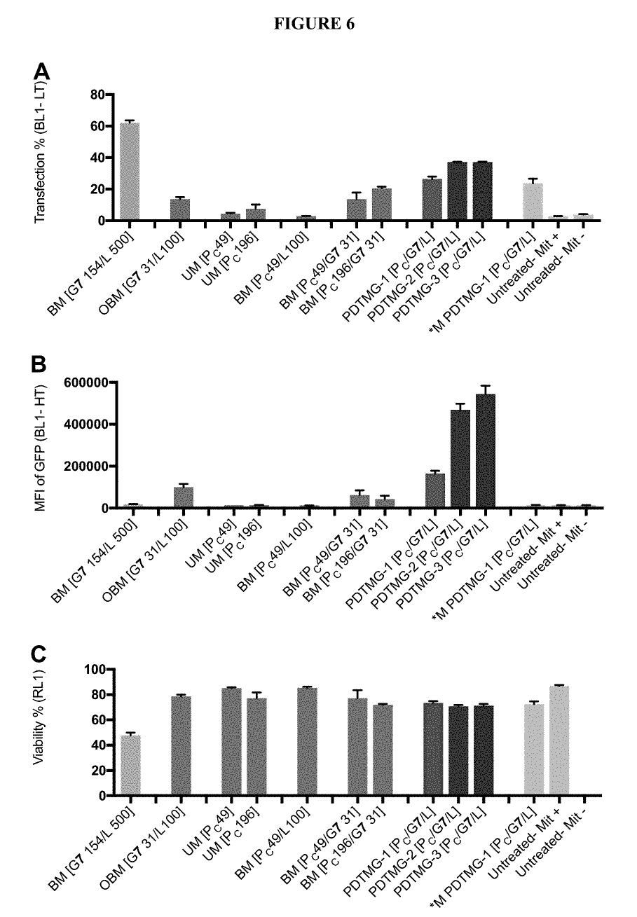

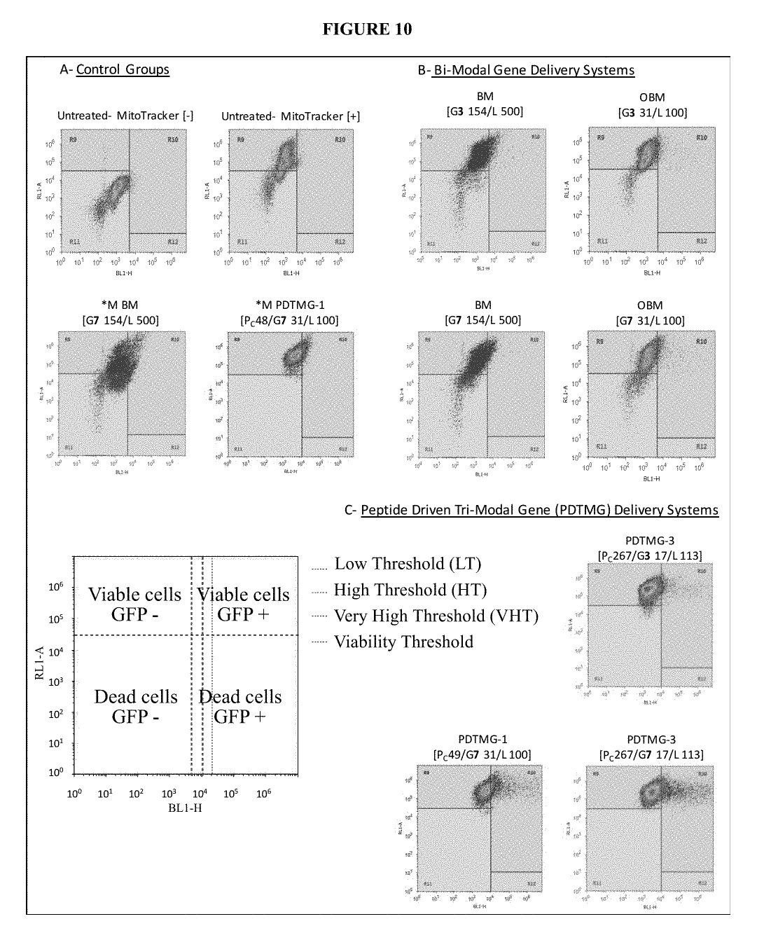

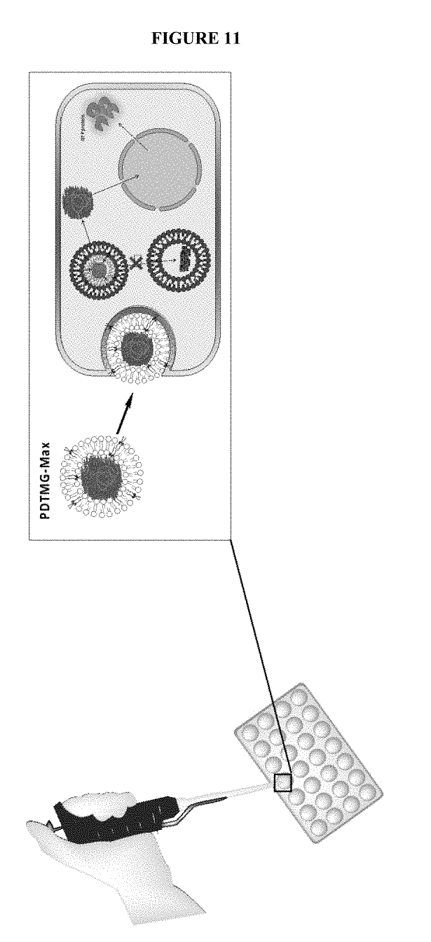

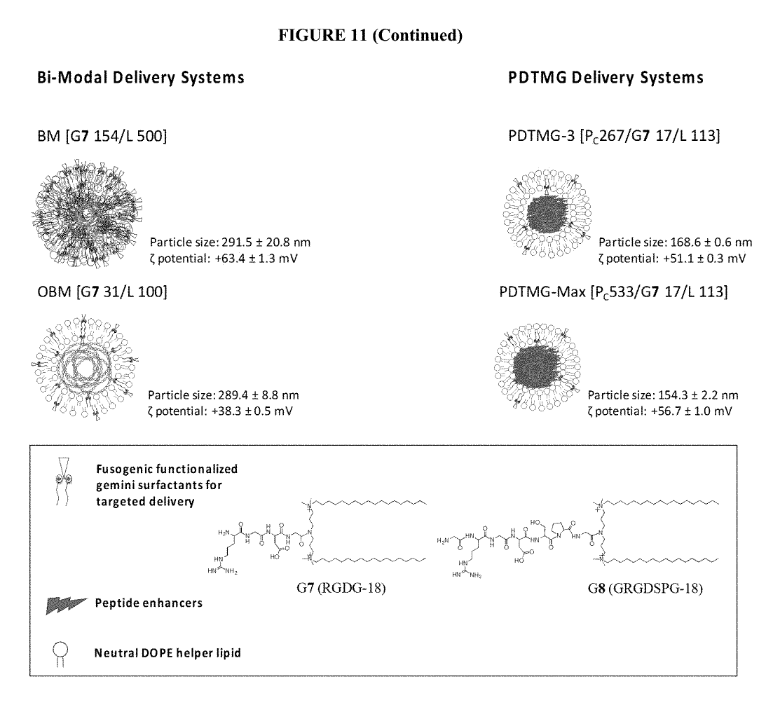

In yet further embodiments, the tri-modal nucleic acid delivery compositions described herein may have a molar concentration of peptide enhancer (M_{P}) of M_{P}≤1000 μM, a molar concentration of gemini surfactant (M_{G}) of M_{G}≤46 μM (such as, for example, 10 μM≤M_{G}≤1000 μM), and a molar concentration of helper lipid (M_{L}) of M_{L}≤300 μM (such as, for example, 30 μM≤M_{L}≤300 μM). To formulate nanoparticles including an adequate amount of each element, the molar concentrations of the compositional elements in the formulation mixtures may be key factors for consideration. Optimization of the compositional elements may include adjustment and balance amongst the three elements to increase the impact of formulations to achieve high transfection efficiency, efficacy and/or cell viability as desired for the particular application. By way of non-limiting example, optimization of a tri-modal gene delivery system comprising [P_{C} cationic peptide enhancers/G7 RGDG-functionalized gemini surfactants/DOPE helper lipids] was achieved by increasing the molar concentrations of biodegradable and non-toxic P_{C} cationic peptide enhancer from M_{P}=49 μM to M_{P}=267 μM and fine tuning of the lipid molarity by reducing the molar concentrations of G7 gemini surfactants from M_{P}=31 μM to M_{P}=17 μM and increasing the molar concentrations of DOPE helper lipids from M_{P}=100 μM to M_{P}=113 μM (FIGS. 6 and 10: optimization of a tri-modal gene delivery system: PDTMG-1 [P_{C}49/G7 31/L 100] vs. PDTMG-3 [P_{C}267/G7 17/L 113]). As illustrated in FIG. 11 (PDTMG-3), the great amount of the cationic peptide enhancers binding to pDNA is encapsulated by a thin lipid membrane made up of an optimized amount of the gemini surfactants and helper lipids, and these together may provide a firm and stable platform for the R functional moieties with a reduced steric hindrance structure (e.g., R₃, R₄) to perform destabilizing movements in response to cellular environment; hence, rupturing the endosome and effectively releasing pDNA into the cell. Without wishing to be bound by theory, it is contemplated that this may at least partially contribute to the remarkable effects in terms of cellular and intracellular delivery of pDNA as observed in the Examples described in detail hereinbelow.

In still further embodiments, the tri-modal nucleic acid delivery compositions described herein may have a surface charge potential) of −60 mV≤ζ≤60 mV. As will be recognized, surface charge may improve the particle stability and impact on transfection efficiency and efficacy of tri-modal gene delivery systems for in vitro and/or in vivo applications. In general, both negatively charged and positively charged particles may improve the stability of the particles. In certain embodiments, the internalization of the nucleic acids (i.e., pDNA) may be facilitated by the positively charged particles or by the neutral or negatively charged particles in the presence of targeting peptide ligands such as transferrin, epidermal growth factor, and/or cell adhesion molecules [1, 2] for site-specific internalization. In general, 18-series cationic gemini surfactants were observed to form more stable particles with higher surface charge as compared to 12-series cationic gemini surfactants at the equal molar ratio. In addition, cationic peptide enhancers also form more stable particles with higher ζ potential as compared to zwitterionic peptide enhancers. In certain embodiments, the ζ potential and stability of the tri-modal nucleic acid delivery systems can, for example, be tuned by balancing the molar concentrations of the cationic gemini surfactants and/or helper lipids and/or the molar concentrations of the peptide enhancers, for example.

In yet further embodiments, the tri-modal nucleic acid delivery compositions described herein may have an average particle size of ≥80 nm and ≤350 nm. In certain embodiments, the size and PDI (polydispersity index) of the particles may correlate with the particle stability, and may impact on transfection efficiency and efficacy of transfection reagents for in vitro and/or in vivo applications. In certain embodiments, the size and/or stability of the particles may be optimized by selection of the compositional elements, and adjustment of their molar concentrations, for example.

As will be understood, in certain embodiments, there is provided herein one or more transfection reagents which may be applicable to a variety of cell lines for delivery of nucleic acids to the targeted cells or tissue for in vitro, ex vivo and/or in vivo (such as, for example, topical) applications. In certain embodiments, there is provided herein transfection reagents which may be designed and developed as a tri-modal nucleic acid delivery platform for targeting of various cell lines in a site-specific manner for in vitro, ex vivo and/or in vivo applications.

An example of a tri-modal delivery composition as described herein may be, for example, PDTMG-Max, which may be used for pDNA delivery into the targeted cells. As shown in Table 4 and further described in the Examples hereinbelow, PDTMG-Max may be formulated from cationic peptide enhancers (P_{B}-P_{G}), RGD-functionalized 18-series gemini surfactants (G7, G8) and DOPE helper lipids (L) at ρ=1.1, r=6.8, M_{P}=533 μM, M_{G}=17 μM and M_{L}=113 μM. By way of non-limiting and illustrative example, PDTMG-Max [PC533/G7 17/L 113] containing 0.5 μg of pDNA in 50 μL formulation mixture may be prepared from aqueous solutions of P_{C} (1 mM stock), G7 (1 mM stock) and L (1 mM stock) according to the following consecutive steps:
mixing 0.5 μg of pDNA with P_{C} cationic peptide enhancer (26.7 μL) and G7 gemini surfactant (0.8 μL);
incubating the pDNA/P_{C}/G7 mixture for 15 minutes at room temperature;
adding the DOPE helper lipids (5.67 μL) to the mixture;
incubating the pDNA/P_{C}/G7/L mixture for 15 min; and
diluting the mixture to a final volume of 50 μL.

As presented in Table 5, PDTMG-Max [P_{C}533/G7 17/L 113] transfection formulation contained nanoparticles with an average size of 154.3±2.2 nm and ζ-potential of +56.7±1.0 mV.

In still another embodiment, there is provided herein a kit for delivering a nucleic acid to a cell, the kit comprising a tri-modal nucleic acid delivery composition including at least one peptide enhancer; at least one surfactant; and at least one helper lipid. As will be understood, each component (i.e. the peptide enhancer, surfactant, and/or helper lipid) may be provided in a separate, unmixed form, or mixed with one or more other components, or with all three components formulated together, for example. The form, state, and degree of mixing for each component may be selected based on the particular application. It is contemplated that the components of the kit may already be in a form suitable for the delivery of nucleic acids, or may be in a pre-formulation state which can be used to generate a formulation suitable for the delivery of nucleic acids. In certain embodiments, the peptide enhancer, surfactant, and/or helper lipid may be each contained in a separate vessel or compartment of the kit, to be later mixed by a user, for example. In certain embodiments, the kits described herein may optionally additionally include instructions for formulating the nucleic acid to be delivered with the tri-modal nucleic acid delivery composition.

In certain embodiments, there is provided herein a method of delivering a nucleic acid to a cell, said method comprising:
generating a delivery vehicle comprising the nucleic acid by formulating the nucleic acid with the tri-modal nucleic acid delivery composition as described herein; and
administering the delivery vehicle to the cell.

In certain embodiments, the generating step may comprise steps of:
mixing the nucleic acid with the peptide enhancer and the surfactant to form a first mixture;
incubating the first mixture for a first incubation time to form a pre-complexed mixture;
adding the helper lipid to the pre-complexed mixture; and
incubating the pre-complexed mixture for a second incubation time to form a complexed mixture.

In certain embodiments, one or both steps of incubating may be performed at about room temperature.

In certain embodiments, the generating step may further comprise an additional step of diluting the complexed mixture to a final volume for use in the administering step.

In certain embodiments, the first incubation time, the second incubation time, or both, may be at least about 5 minutes, at least about 10 minutes, or at least about 15 minutes, based on the concentration being used, where incubation times may be shorter for higher concentration mixtures.

In certain embodiments, the methods may be performed in vitro (for example, on cells in culture), or in vivo (i.e. on a subject in need thereof). For in vitro methods, administration may include incubating the cells with the nucleic acid delivery composition comprising the nucleic acid. For in vivo methods, administration may include local or systemic administration using any suitable administration route such as, but not limited to, topical, oral, subcutaneous, intramuscular, intranasal, intravenous, intraperitoneal injection, or local injection administration. Administration may, in certain embodiments, involve microneedle, dropper, spray applicator, nebulizer, syringe, or other suitable administration techniques or devices. The skilled person having regard to the teachings herein will recognize suitable administration routes and techniques/devices to suit a particular application.

As will be understood, in certain embodiments the tri-modal nucleic acid delivery compositions described herein may be used for delivery to a wide variety of cells, at least particular due to surface charge (which, in some examples, was measured to be around +60 mV). As will be understood, target or specific cell delivery to target cell-types is also contemplated herein, and may be performed using, for example, targeting moieties functionalized to the delivery systems.

In certain embodiments, there is provided herein a method of preparing a nucleic acid for delivery to a cell, said method comprising:
formulating the nucleic acid with a tri-modal nucleic acid delivery composition as described herein.

By way of a non-limiting and illustrative example, as shown in Table 4, the desired amount of pDNA (e.g., 0.1 μg, 0.5 μg, 2.5 μg, 5 μg, 25 μg etc., based on the targeted area for DNA transfection) may be formulated using, for example, PDTMG-Max tri-modal gene delivery systems as described herein. By way of non-limiting example, the compositional elements of PDTMG-Max may include P_{B}-P_{G} cationic peptide enhancers, G7 or G8 gemini surfactants, and DOPE helper lipids, and may formulate pDNA at ρ=1.1, r=6.8, MP=533 μM, MG=17 μM and M_{L}=113 μM. By way of example, such formulation may include:
mixing the nucleic acid with the peptide enhancer and the surfactant to form a first mixture;
incubating the first mixture for a first incubation time to form a pre-complexed mixture; and
adding the helper lipid to the pre-complexed mixture; and
incubating the pre-complexed mixture for a second incubation time to form a complexed mixture.

In certain embodiments, one or both steps of incubating may be performed at about room temperature.

In certain embodiments, the formulating step may further comprise an additional step of diluting the complexed mixture to a final volume.

In certain embodiments, the first incubation time, the second incubation time, or both, may be at least about 5 minutes, at least about 10 minutes, or at least about 15 minutes, based on the concentration being used, where incubation times may be shorter for higher concentration mixtures.

While cellular uptake of DNA is an important criterion for an efficient gene delivery system, transfection efficacy is reliant on critical endosomal escape. The following examples describe detailed experimental studies in which several nucleic acid delivery compositions were designed and subjected to in-depth study. As part of the following research, eleven distinct gemini surfactants were designed and synthesized by covalent linking of 10 different functional moieties (R₁-R₁₀) [imidazole- and thiol-containing functional groups (R₁, R₂), and linear RGD peptides (R₃=RGDG (SEQ ID NO: 8), R₄=GRGDSPG (SEQ ID NO: 9), R₆=EGRGDSPG(H)₅ (SEQ ID NO: 10))] to the spacer regions of m-7NH-m gemini surfactants (m-s-m formula; m=12, 18 carbon alkyl chains, s=imino-substituted-7 methylene spacer group).

In a further part of the research described below, the RGD-functionalized gemini surfactants were evaluated for targeted gene delivery. As well, the impact of non-covalent addition of designed zwitterionic or cationic peptide enhancers were examined for development of gene delivery systems carrying, in these examples, green fluorescent protein (GFP)-expressing plasmid DNA (pDNA). Among fourteen different gemini surfactants [G1-G14 (m=12, 18 and s=3, 7NH, 7NR₁₋₁₀)], remarkably compounds G7 (18-7N(R₃)-18) and G8 (18-7N(R₄)-18) formulated peptide driven tri-modal gene delivery systems (PDTMG), comprising [cationic peptide enhancers/gemini surfactants/1,2-dioleyl-sn-glycero-3-phosphoethanolamine (DOPE) helper lipid], provided both elevated cell-penetrating activity and endosomal rupturing functionality in the experiments performed as detailed hereinbelow.

Without wishing to be bound by theory, it is believed that the short RGD functional peptides (R₃, R₄) linked to 18-series gemini surfactants provided reduced steric hindrance on the surface of the PDTMG nanoparticles and exhibited endosomal destabilizing effects in response to cellular environment. The non-covalent addition of cationic peptide enhancers formulated in the PDTMG delivery systems demonstrated a remarkable multicomponent system for effective nucleic acid (in this example, DNA) condensation, particle stability, cellular uptake, amplified endosomal release, protecting and facilitating the intracellular delivery of the pDNA in these experiments. Results detailed in the examples hereinbelow indicate that a variety of nucleic acid delivery compositions have been developed which may provide notable transfection efficiency and efficacy properties. In particular, the potent virus-like nanoparticles G7 or G8 formulated PDTMG offered a versatile delivery system for targeted delivery of nucleic acids, such as nucleotide-based therapeutics, and suggest applicability even to in vivo nucleotide-based gene therapy and/or DNA vaccine applications, for example.

### EXAMPLES

### Preparation and Testing of Nucleic Acid Delivery Compositions

The following studies describe a detailed research and development program aimed at developing potent nucleic acid delivery systems and surfactants for the delivery of nucleic acids. As part of these studies, cationic gemini surfactants were developed and employed. Gemini surfactants are a group of surfactants made up of two monomeric surfactants linked together by a spacer group [8-11]. In certain embodiments, gemini surfactants may include those having the formula:

m-s-m (formula I);
wherein each m independently represents the number of alkyl tail carbon atoms of each respective monomeric surfactant portion, and s represents the number of atoms in the spacer group linking the two surfactant portions.

By way of example, a gemini surfactant of formula II may be considered as an embodiment of a gemini surfactant of formula I as follows:

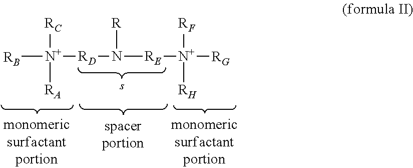

wherein at least one of R_{A}, R_{B}, and R_{C} comprises an alkyl-based tail having m carbon atoms, and the remaining of R_{A}, R_{B}, and R_{C} are substituents, such as alkyl (for example, C₁-C₄ alkyl) substituents or an imidazole-based or thiol-based or hydroxyl-based group (for example), which cause the nitrogen to which they are attached to be quaternary;
wherein at least one of R_{F}, R_{G}, and R_{H} comprises an alkyl-based tail having m carbon atoms (which may be the same, or different, from m defined above), and the remaining of R_{F}, R_{G}, and R_{H} are substituents, such as alkyl (for example, C₁-C₄ alkyl) substituents or an imidazole-based or thiol-based or hydroxyl-based group (for example), which cause the nitrogen to which they are attached to be quaternary; and
wherein the spacer —R_{D}—N(R)—R_{E}— links the two monomeric surfactant portions through their respective quaternary nitrogens, R_{D} and R_{E} each represent an alkyl-based group or derivative thereof, R represents a functional moiety joined to the nitrogen of the spacer, and s represents the total number of spacer atoms along the shortest linear path running between the quaternary nitrogens.

By way of further example, a gemini surfactant of formula III may be considered as an embodiment of a gemini surfactant of formulas I and II as follows:

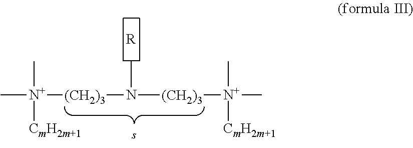

As shown above, formula III is characterized by the structure m-s-m of formulas I and II, wherein two quaternary nitrogen-based surfactants, each having two methyl groups and an alkyl tail on their quaternary nitrogens, are linked together via a —CH₂—CH₂—CH₂—N(R)—CH₂—CH₂—CH₂— spacer (abbreviated 7NR, where s is 7) joining the two quaternary nitrogens. The experimental examples described below typically employ cationic gemini surfactants of formula III wherein m is 12 or 18 and s is 7 (as shown if formula III), and R is selected from R₁-R₁₀ as shown in FIG. 1. In certain embodiments, cationic gemini surfactants may include those based on N,N-bis(dimethylalkyl)-α,ω-alkanediammonium.

Gemini surfactants have been shown to provide high levels of interfacial activity and promote self-assembly at concentrations about a hundredfold lower as compared to the corresponding monomeric surfactants [12-17]. Cationic gemini surfactants formulated with neutral helper lipids, such as DOPE (1,2-dioleyl-sn-glycero-3-phosphoethanolamine) and DPPC (1,2-dipalmitoyl-sn-glycero-3-phosphocholine), have been widely used as non-viral gene delivery systems [18-22]. Through chemical modification of the spacer group and the alkyl chains, further compounds can be designed to improve specific DNA transfection [23]. The substitution of the alkyl spacer with pH sensitive imino groups was developed to increase the transfection efficiency of gene delivery systems [14-16]. The covalent grafting of linear RGD derivatives (GRGDSP) to dioleyl lipid tails via PEG2000, and coupling of cyclic RGD peptide (cRGDfK) to 12-series gemini surfactants separated by two ethylene oxide units were performed to target genes for integrin-mediated internalization [24, 25]. RGD (arginine-glycine-aspartic acid) peptidomimetics bind to integrin receptors on melanoma, fibroblasts and epithelial cells and are believed to have broad application to target drugs and genes to specific cells [1, 26, 27].

In the presently described studies, the effect of covalent functionalization of spacer regions of m-7NH-m gemini surfactants (m=12 and 18 carbon alkyl chains, s=imino-substituted-7 methylene spacer group) with 10 different functional moieties R₁-R₁₀ (see FIG. 1) [imidazole and thiol containing functional groups (R₁=imidazolpropionyl, R₂=thiopropionyl), linear RGD derivatives (R₃=RGDG (SEQ ID NO: 8), R₄=GRGDSPG (SEQ ID NO: 9)), polyhistidine derivatives (R₅=E(H)₅ (SEQ ID NO: 11)), bifunctional RGD-polyhistidine peptides (R₆=EGRGDSPG(H)₅ (SEQ ID NO: 10)), zwitterionic and cationic arginine rich peptide motifs (R₇=Suc-(E)₂G(R)₂ (SEQ ID NO: 12), R₈=Suc-(E)₂G(R)₃ (SEQ ID NO: 13), R₉=Suc-(E)₂(G)₃(R)₃ (SEQ ID NO: 14) and R₁₀=Suc-DE(G)₃(R)₃) (SEQ ID NO: 15)] have been investigated for pDNA delivery. Further, the impact of non-covalent addition of peptide enhancers (P_{A}-P_{G}; see Table 3) with various charges and different lengths consisting of histidine and/or arginine residues and/or RGD motifs (GRGDSP; SEQ ID NO: 16) were examined for development of potent gene delivery systems.

In vitro transfection efficiency, efficacy, and cell viability of various nucleic acid delivery formulations containing pDNA encoding green fluorescent protein (GFP) (see Table 6 and Table 7) using fourteen different gemini surfactants (m-s-m formula; m=12 and 18 carbons alkyl lengths, s=3, 7NH and 7NR₁₋₁₀ spacer groups) and seven peptide enhancers have been evaluated using 3T3-Swiss albino mouse fibroblasts by flow cytometry. Using quantitative flow cytometry, outlining parameters were created to provide distinct information on both transfection efficiency and efficacy of the delivery systems. The correlation of the transfection efficiency and efficacy to the physicochemical properties of delivery systems were identified to advance formulation strategies for development of a potent delivery system.

Enhanced multicomponent peptide driven tri-modal gene delivery systems (PDTMG) consisting of [peptide enhancers/gemini surfactants/DOPE helper lipids] were developed through various formulation strategies. These include optimization of gemini/DNA charge ratio (ρ values), DOPE/gemini molar ratio (r values), and the molarity of the compositional elements in the formulation mixtures (M_{P}, M_{G} and M_{L} for molar concentrations of peptide enhancers (P), gemini surfactants (G) and DOPE helper lipids (L), respectively).

The following studies report the results of multifactorial considerations for development of, for example, virus-like nanoparticles, RGD-functionalized gemini surfactants, and formulated PDTMG for targeted gene delivery.

Experimental Procedures

Materials

Custom designed peptide enhancers (7 types: P_{A} (GRGDSPG; SEQ ID NO: 1); P_{B} (H(R)₃H(R)₃HG; SEQ ID NO: 2); P_{C} (GRGDSPGH(R)₃H(R)₃HG; SEQ ID NO: 3); P_{D} ((H)₅; SEQ ID NO: 4); P_{E} (GRGDSPG(H)₅; SEQ ID NO: 5); P_{F} ((H)₂R(H)₇R(H)₃G; SEQ ID NO: 6); P_{G} (GRGDSPG(H)₂R(H)₇R(H)₃G; SEQ ID NO: 7)) were purchased from Biomatik Corporation (Cambridge, ON, Canada) (purity>95%). 1-N-trityl-imidazole-2-ylpropionic acid and 3-(tritylthio)propionic acid (protected R₁ and R₂ functional moieties, respectively) were obtained from Sigma-Aldrich (Oakville, ON, Canada). The protected peptide functionalities (R₃-R₁₀) were purchased from Biomatik Corporation (Cambridge, ON, Canada). The resin-cleaved protected R₃ (Boc-Arg(Pbf)-Gly-Asp(OtBu)-Gly-OH) and R₄ (Boc-(Gly)-Arg(Pbf)-Gly-Asp(OtBu)-Ser(tBu)-Pro-Gly-OH) were obtained with the free C-terminal carboxylic groups (purity>95%). The rest of protected functionalities (R₅-R₁₀) were acquired on resin with the free N-terminal carboxylic groups. The protected R₅ (Boc-Glu-(His(Trt))₅) and R₆ (Boc-Glu-Gly-Arg(Pbf)-Gly-Asp(OtBu)-Ser(tBu)-Pro-Gly-(His(Trt))₅) were obtained on H-His(Trt)-2-Chlorotrityl Resin (0.342 mmol/g); while the protected R₇ (succinyl-Glu(OtBu)-Glu(OtBu)-Gly-Arg(Pbf)-Arg(Pbf)), R₈ (succinyl-Glu(OtBu)-Glu(OtBu)-Gly-Arg(Pbf)-Arg(Pbf)-Arg(Pbf)), R₉ (succinyl-Glu(OtBu)-Glu(OtBu)-Gly-Gly-Gly-Arg(Pbf)-Arg(Pbf)-Arg(Pbf), and R₁₀ (succinyl-Asp(OtBu)-Glu(OtBu)-Gly-Gly-Gly-Arg(Pbf)-Arg(Pbf)-Arg(Pbf)) were procured on Rink amide MBHA Resin (0.45 mmol/g or 0.342 mmol/g)). All chemicals including 1-[bis(dimethylamino)methylene]-1-H-1,2,3-triazolo[4,5-b]pyridimium 3-oxid hexafluorophosphate (HATU), N,N-diisopropylethylamine (DIPEA), trifluoroacetic acid (TFA), triisopropylsilane (TIS), 1,2-ethanedithiol (EDT), N,N-demethylformamide (DMF) and HPLC grade acetonitrile (MeCN) were purchased from Sigma-Aldrich (Oakville, ON, Canada). Analytical ultra-performance liquid chromatography (UPLC) was performed on a Waters ACQUITY UPLC H-Class BioSystem (Milford, Mass., USA) with a flow rate of 0.2 mL/min and UV detection at 214 nm. Semi-preparative reverse phase high performance liquid chromatography (RP-HPLC) was performed on a Waters instrument (Waters e2695 separations module) (Milford, Mass., USA) at a flow rate of 10 mL/min and UV detector set to a wavelength of 214 nm. The mobile phases for both analytical UPLC and semi-preparative HPLC were solvent A (water/TFA: 99.9/0.1, v/v) and solvent B (MeCN/TFA: 99.9/0.1, v/v). Analytical separation was achieved by a linear gradient of solvent B on ACQUITY UPLC BEH C18 column (130 Å pore size, 1.7 μm particle size, 2.1 mm×50 mm); while, the semi-preparative separation was on 300SB-C18 semi-preparative column (300 Å pore size, 5 μm particle size, 9.4 mm×250 mm). Electrospray ionization mass spectrometry (ESI-MS) was performed on a Q-Exactive Orbitrap System (Thermo Fisher Scientific, CA, USA) using a mixture of solvent A (water/formic acid, 99.9/0.1, v/v) and solvent B (MeCN/formic acid, 99.9/0.1, v/v).

Synthesis and Purifications of Functionalized Gemini Surfactants

The synthesis of non-functionalized gemini surfactants (G1-G3; m-3-m and m-7NH-m) were carried out according to the previously published procedures [9, 11, 14, 16, 23]. The covalent R-functionalization (R₁-R₁₀; Table 1 and FIG. 1) of m-7NH-m gemini surfactants [G4-G14] were performed either in solution (40 μmol scale in 20 mL of MeCN) (Method A, FIG. 2) or in the solid phase on-resin (at 100-200 μmol scale in 10 mL of DMF) (Method B, FIG. 2) by amide bond formation between the imino spacer of gemini surfactants (1 eq.) and the pre-activated free carboxylic groups of the protected R-functional moieties (2 eq.) using HATU (1.9 eq.) and DIPEA (2.8 eq.). After 3-4 hours' completion of the ligation reactions, the cleavage/deprotection step was accomplished using a cocktail of TFA/Water/TIS (95:2.5:2.5) (for compounds G4, G6-G14) or TFA/thioanisol/EDT/anisole (90:5:3:2) (for compounds G5) or over 2-3 hours. Crude products were purified by semi-preparative RP-HPLC (Table 2), lyophilized and kept at −20° C. The quantitative and qualitative identification of the synthesized compounds confirmed by analytical RP-UPLC and electrospray ionization-mass spectrometry (ESI-MS) (purity>95%).

Preparation of Formulations

The freshly made stock solution of DOPE (L) helper lipids (Avanti Polar Lipids, Alabaster, Ala., USA) were prepared at 1 mM concentration in sucrose solution (9.25% w/v) by bath sonication (10 min) and high-pressure LV1 Microfluidizer (×3 at 20,000 psi) as described previously [28, 29]. The aqueous solutions of gemini surfactants (G1-14) and peptide enhancers (P_{A}-P_{G}) were separately prepared in nuclease-free water. Uni-Modal (UM [P], UM [G]), Bi-Modal (BM [G/L], BM [P/L], BM [P/G]) and Tri-Modal (PDTMG [P/G/L]) delivery systems (Table 6 and 7; 54 formulation types) were formulated at various ρ and r values, and molar concentrations (M_{P}, M_{G}, M_{L}; see Table 4 for detailed information on the selected formulations). The formulation mixtures were pre-incubated for 30 minutes at room temperature before being used in the transfection assay. The gWiz™ GFP pDNA (5757 bp; Aldevron, Fargo, N. Dak., USA) was used to monitor the expression level of the reporter genes. A mock pDNA (5688 bp; Blue Heron Biotech, Bothell, Wash., USA) with absent of a fluorescent protein reporter gene was used to control the transfection efficacy of the formulations. The commercially available Lipofectamine™ 3000 reagent (Invitrogen Life technologies) was used as a reference transfection reagent according to the manufacturer's instructions.

By way of a non-limiting example, PDTMG-Max [P_{C}533/G7 17/L 113] gene delivery system may be used to formulate required amount of pDNA for transfecting cells (pDNA: 0.1 μg, 0.5 μg or 2.5 μg in 10 μl, 50 μL or 250 μL transfection formulations, respectively) at ρ=1.1, r=6.8, M_{P}=533 μM, M_{G}=17 μM and M_{L}=113 μM as presented in Table 4.

The PDTMG-Max [P_{C}533/G7 17/L 113] delivery formulation containing 0.5 μg of pDNA can be prepared using a transfection kit containing [Tube A (P_{C} cationic peptide enhancers at 1 mM concentration), Tube B (G gemini surfactants at 1 mM concentration) and Tube C (DOPE helper lipids at 1mM concentration)] according to the following 5 consecutive steps:
1—add 26.7 μL from Tube A and 0.8 μL from Tube B in a microtube containing 0.5 μg of pDNA, and mix well,
2—incubate the mixture for 15 minutes at room temperature,
3—add 5.67 μL from Tube C to the mixture and mix well,
4—incubate the mixture for 15 min,
5—dilute the mixture to a final volume of 50 μL.

Physicochemical Characterization of Formulations

The gene delivery formulations were prepared as described above. Size measurements were performed at the same concentration used in the transfection assay; while, zeta (ζ)-potential measurements were performed by diluting samples to a final volume of 1 mL in nuclease-free water. The size (mean hydrodynamic diameters) and ζ-potential of the particles were measured at 25° C., with a 1 min equilibrium time, and automatic measurement cycle using Zetasizer Nano ZS instrument (Malvern instruments Ltd., Worcestershire, UK). Data points are the average of three measurements (n=3)±standard deviation (SD).

Cell Culture and In Vitro Transfection

Mouse fibroblasts 3T3-Swiss albino (ATCC® CCL-92TM) were cultured in Dulbecco's modified Eagle's medium (DMEM)—high Glucose supplemented with 10% fetal bovine serum and 1% penicillin/streptomycin and incubated at 37° C. under an atmosphere of 5% CO₂. Cells were seeded in 96-well/24-well tissue cultured plates (Corning Inc., Corning, N.Y., USA) at a density of 15,000 cells/cm². After 24 h (when 85-90% confluency was achieved) and 1 h prior to transfection, the complete medium was replaced with the basic DMEM medium without serum and antibiotic. Cells were transfected with formulations containing pDNA (0.1 μg/well of 96-well plate or 0.5 μg/well of 24-well plate) and incubated at 37° C. for 5 h. The fresh complete growth medium was added to each well without removing transfection formulations and further incubated for 19 h. After 24 h of transfection, cells were trypsinized and stained with MitoTracker Deep Red (0.5 μL/mL) for 15 min at 37° C. Transfection efficiency (presented by the percentage of the pDNA-transfected cells), efficacy (expressed by the mean fluorescence intensity (MFI) of the cells expressing GFP), and cell viability were examined by flow cytometry (Attune® Flow Cytometer, Life Technologies, Carlsbad, Calif., USA).

Transfection Study and Cell Viability by Flow Cytometry

To create a consistent flow cytometry analysis, flow cytometry parameters were adjusted according to fluorescent and non-fluorescent cells prepared by electroporation with PmaxGFP™ reporter pDNA or mock pDNA using Lonza Nucleofector Kit (Lonza Inc., Basel, Switzerland). Cell viability of the transfected cells were measured by assessing the metabolic activity of the mitochondria, stained with MitoTracker Deep Red as previously described [29]. The intensity of green fluorescence vs. MitoTracker stain signals were used to assess the impact of transfection reagents on cell expressions and viability of transfected cells. The expressions of the fluorescent proteins were detected in the BL1 channel (emission filter: 530/30 nm for GFP detection) using 488 nm blue laser as an excitation source. MitoTracker Deep Red mitochondria stain was excited with 638 nm red laser and detected in RL1 channel (emission filter: 650-670 nm). FSC (forward scatter) and SSC (side scatter) voltages were set at 1350 (mV) and 2400 (mV), respectively, to place the events in the appropriate area in the FSC vs. SSC dot plot. The thresholds for BL1 and RL1 fluorescence channels were adjusted to 1450 (mV) and 1400 (mV), respectively, to locate the cell population in the two-dimensional (2D) density plot of the BL1 vs. RL1. A total number of 20,000 cell events were recorded for cell cycle analysis. The cell viability index was calculated as follows:

(V_{treated sample}/V_{untreated control})×100%

Statistical Analysis

All data are presented as means±SD (n≥2) and in vitro studies of the samples were performed in at least 2 independent experiments to ensure reproducibility. Differences between groups were identified by one-way analysis of variance (ANOVA) followed by Tukey's multiple comparison post-hoc test. GraphPad Prism (version 7.0c, GraphPad Software, Inc.) was used for statistical analyses. Statistical significant differences were considered when P<0.05.

Results

Design and Synthesis of Functionalized-Gemini Surfactants

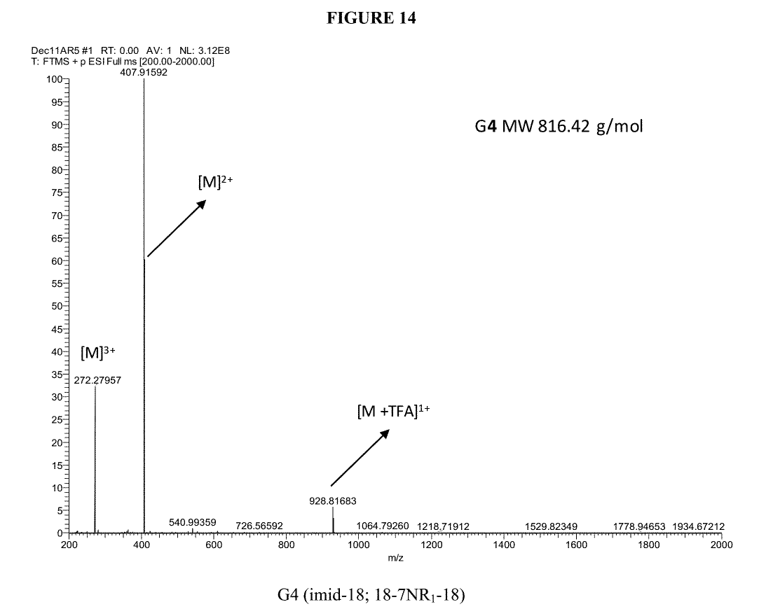

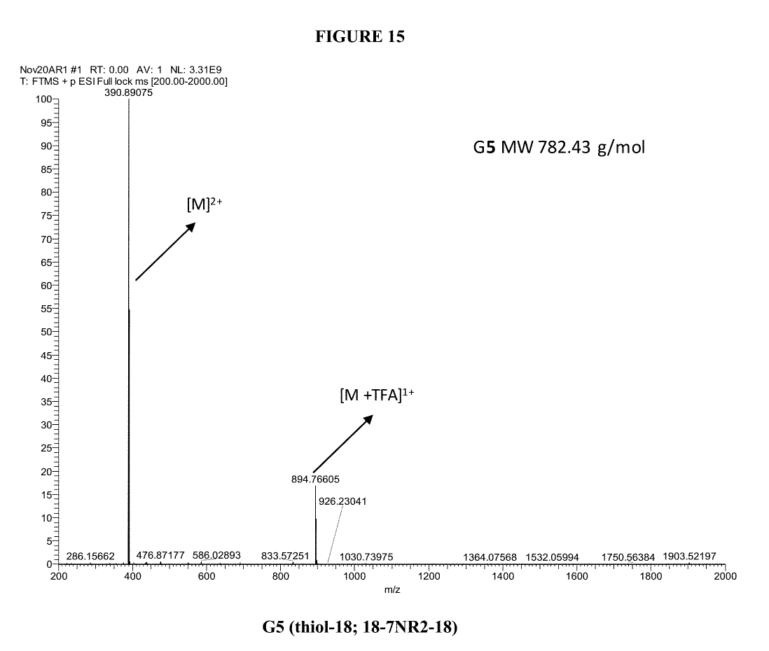

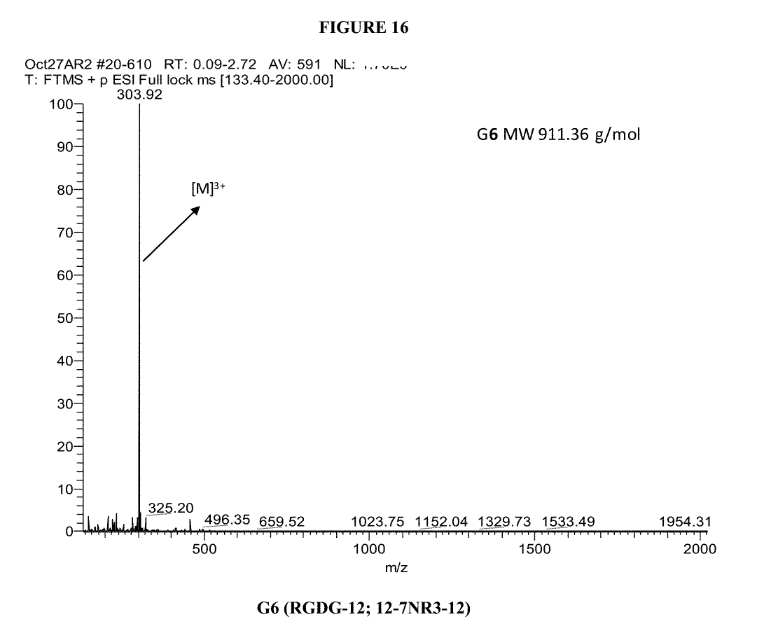

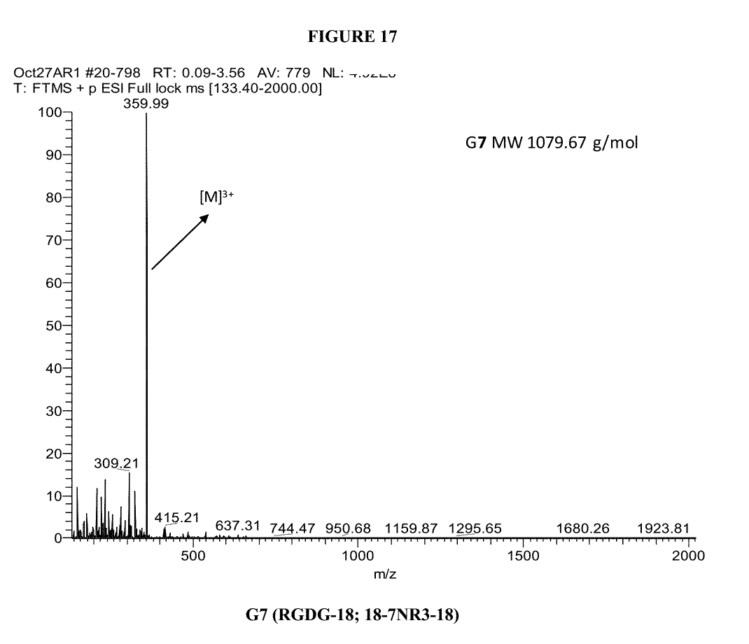

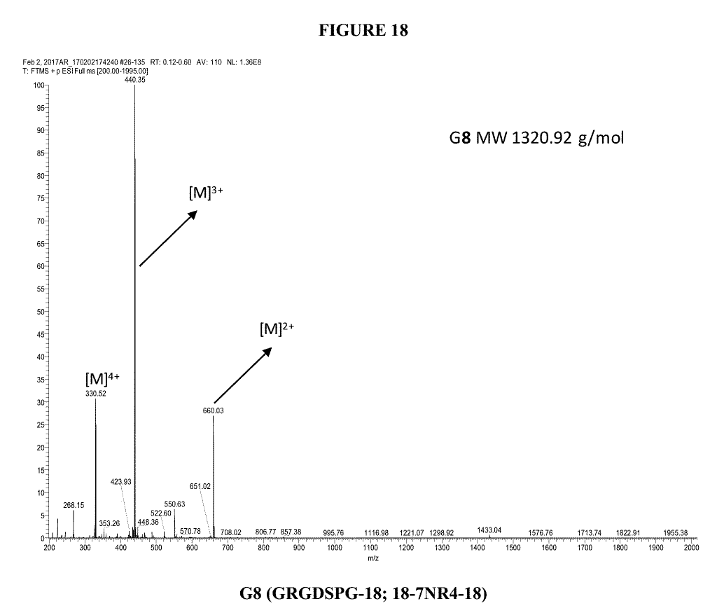

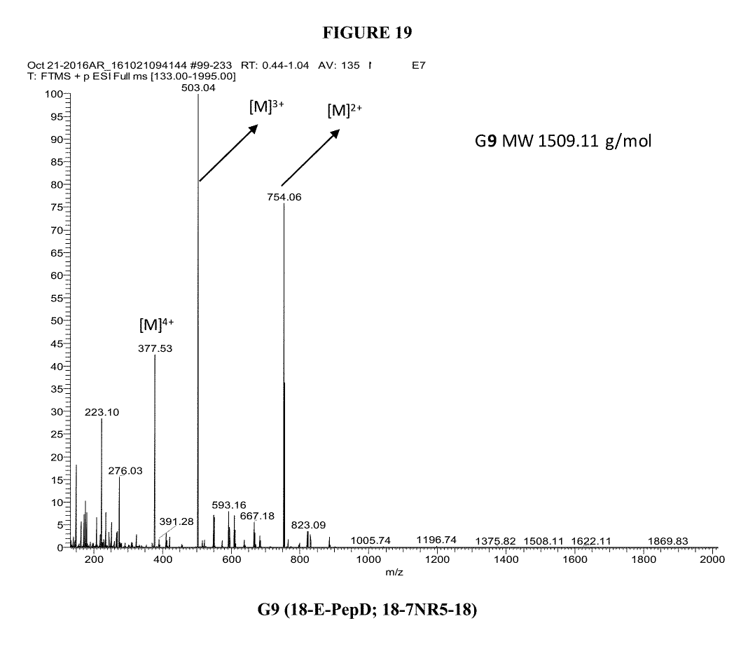

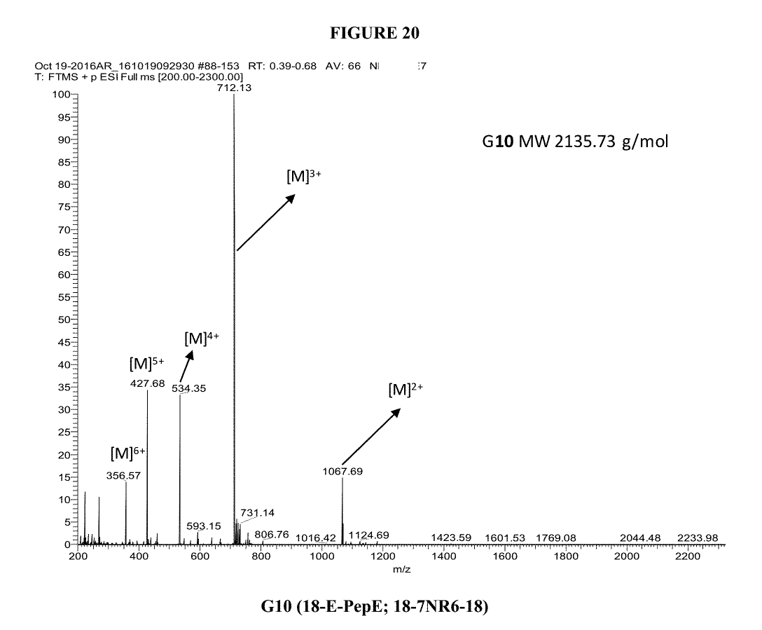

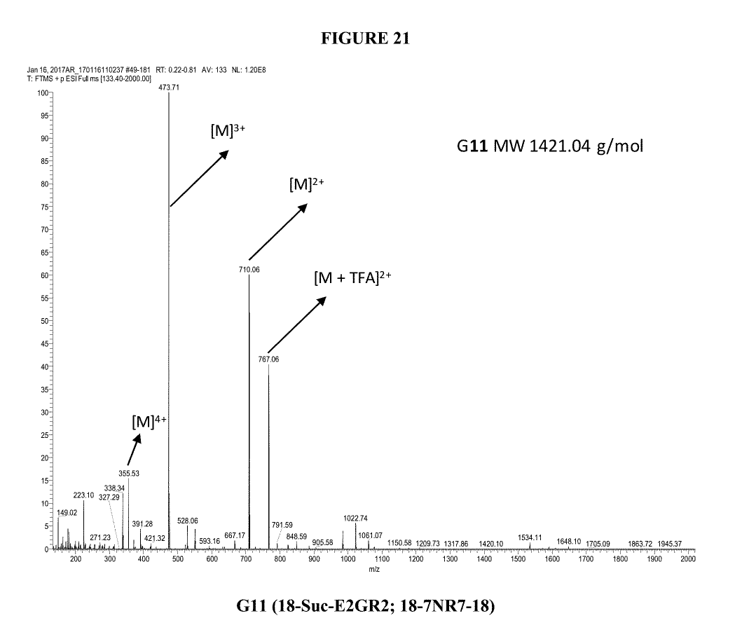

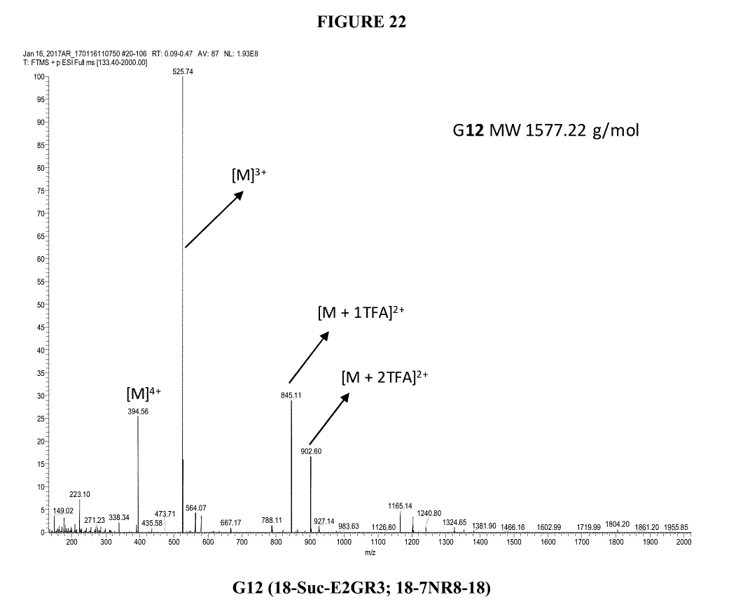

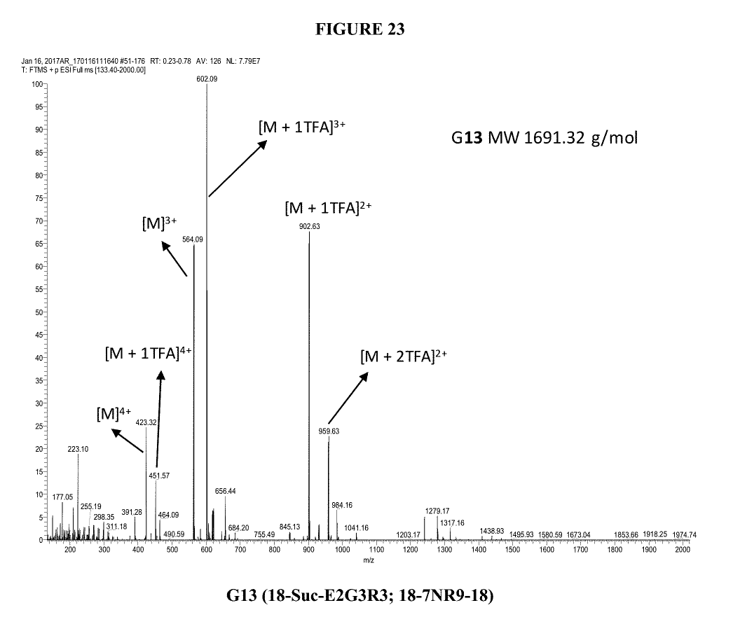

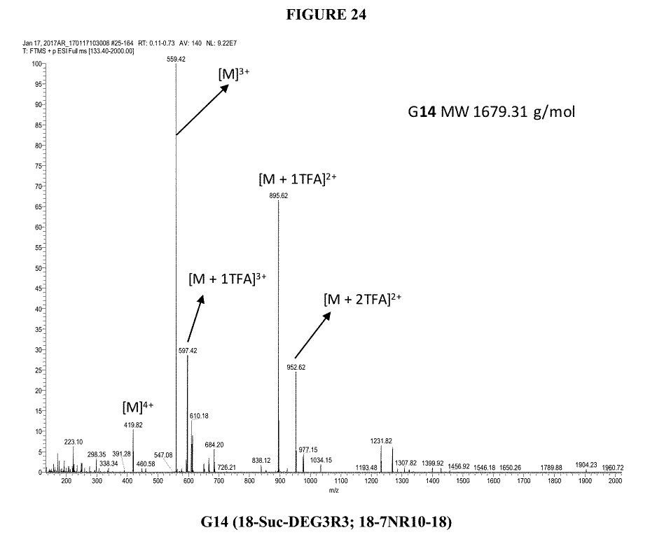

Eleven novel functionalized gemini surfactants [G4-G14] (m-7NR-m formula; m=12, 18 and R=R₁-R₁₀; Table 1, FIGS. 1 and 13) were co-designed and synthesized. R-functionalization of the gemini surfactants was carried out either by Method A in solution for compounds G4-G8 (FIG. 2(A)) or by Method B in the solid phase for compounds G9-G14 (FIG. 2(B)). The imino group of the m-7NH-m gemini surfactants were conjugated to free carboxylic groups located either at the C-terminus of the protected functional moieties [R₁-R₄] using Method A or at the N-terminus of the protected functional peptides [R₅-R₁₀] using Method B (FIG. 13). The synthesized products were purified to a single peak (purity>95%) using analytical reverse phase high performance liquid chromatography (RP-HPLC) and the identity of the products were confirmed by ESI-MS (Table 2, and FIGS. 14-24).

Particle Size and Zeta Potential Analysis

The physicochemical properties of gene delivery formulations in correlation with their transfection efficiency and efficacy profile were analyzed to advance formulation strategies for development of a potent delivery system. The hydrodynamic diameters, polydispersity index (PDI) and surface charge (ζ-potential) of various gene delivery formulation types (i.e., UM [P], UM [G], BM [G/L], BM [P/L], BM [P/G], PDTMG [P/G/L]) were characterized by dynamic light scattering (DLS) (Table 5). To put the formulation possibilities into perspective, the characterization of 37 selected formulations (Table 5) are discussed in this report to provide a general insight in order to predict optimized formulations for pDNA delivery. DNA transfection generally follows these steps: first, effective compaction of negatively charged pDNA into stable positively charged particles (or neutral particles in the presence of targeting moieties) to facilitate cellular uptake across the negatively charged cell surface membrane; second, endosomal release and protection of pDNA against intracellular degradation, and eventually, nuclear translocation.

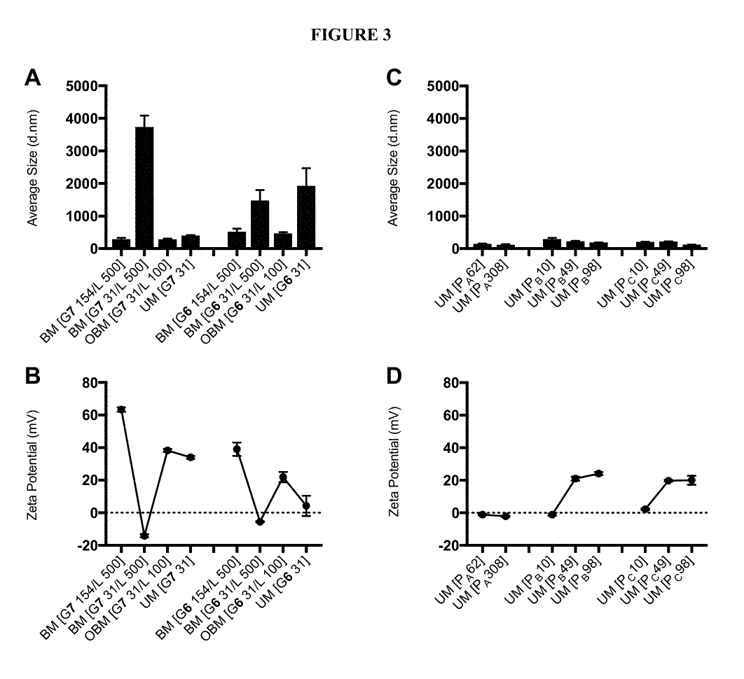

The physicochemical characterization of UM [G] and BM [G/L] gene delivery systems were investigated using RGDG-18 (G7; 18-7N(RGRG)-18) and RGDG-12 (G6; 12-7N(RGRG)-12) gemini surfactants and DOPE helper lipids at various lipid molarity (M_{G}=154 μM, 31 μM; M_{L}=500 μM, 300 μM, 100 μM) (Table 5). As shown in FIGS. 3(A) and (B), the UM [G] and BM [G/L] gene delivery formulations using 18-series gemini surfactants can generally form smaller particle aggregates, tighter DNA compactions, with higher ζ-potential as compared to 12-series gemini surfactants at the equal molar ratio (e.g., size: 408.1 ±6.8 nm vs. 1925.3 ±271.4 nm; ζ-potential: +34.0±0.5 mV vs. +4.2±3.1 mV for UM [G7 31] and UM [G6 31], respectively). Through both decreasing the M_{G} from 154 μM (ρ=10) to 31 μM (ρ=2) and the M_{L} from 500 μM to 100 μM (r=3.3), the (at least partially) optimized BM gemini/lipid gene delivery systems (OBM [G 31/L 100]) were formulated to form stable and small particles for endosomal release of pDNA (as discussed in further detail below) (e.g., 291.5±20.8 nm vs. 3738.7±172.4 nm vs. 289.4±8.8 nm for BM [G7 154/L 500], BM [G7 31/L 500] and OBM [G7 31/L 100], respectively). This, however, resulted in substantial decrease in the ζ-potentials of BM particles (e.g., +63.4±1.3 mV, −14.1±0.5 mV and +38.3±0.5 mV for BM [G7 154/L 500], BM [G7 31/L 500] and OBM [G7 31/L 100], respectively).

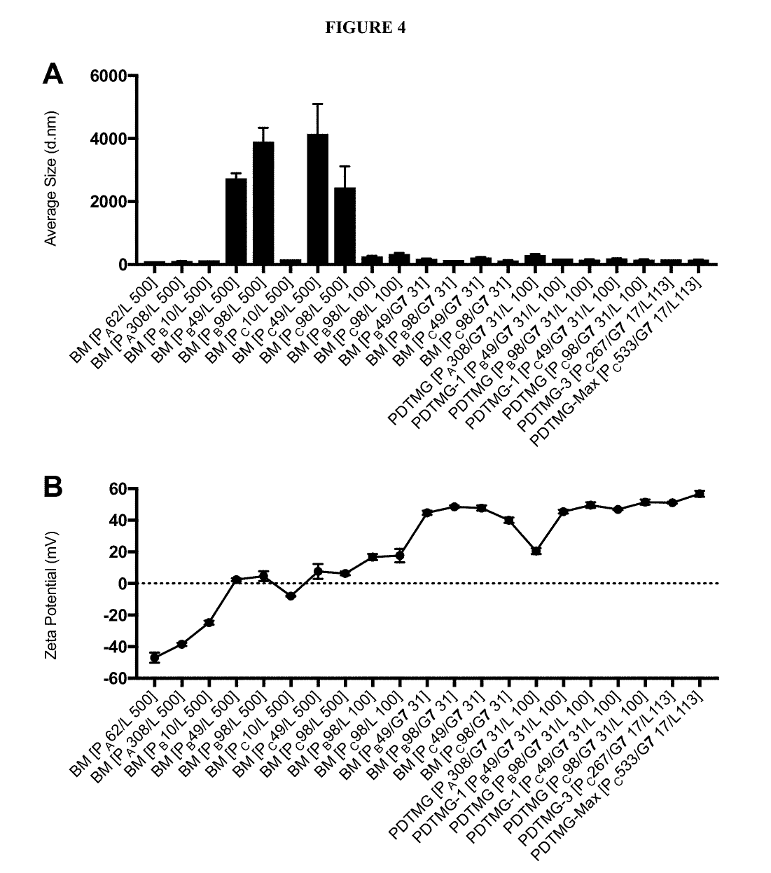

To analyze the effect of non-covalent addition of non-toxic and biodegradable peptide enhancers, first the UM [P] gene delivery formulations were characterized by complexation of pDNA at various peptide molarity (M_{P}) (FIGS. 3(C) and (D)). These peptide enhancers were designed to have various charges (0, 0.5, 3.2, 6.3) and lengths to include histidine and/or arginine residues and/or RGD motif (GRGDSP) (Table 3). It was shown that by increasing the molar concentrations of cationic peptide enhancers (e.g., P_{B} and P_{C}) from 10 μM to 98 μM, the size of the UM [P] formulations decreased; while, their ζ-potentials increased to approximately 20 mV (e.g., +2.2±0.2 mV vs. +20.0±1.4 mV for UM [P_{C}10] and UM [P_{C}98], respectively). Increasing M_{P} for zwitterionic (neutral) peptide enhancers (i.e., P_{A}), however, showed no significant changes in ζ-potentials of the formulated UM [P_{A}] (−1.2±0.2 mV and −2.2±0.2 mV for UM [P_{A}62] and UM [P_{A}308], respectively). The incorporation of biodegradable peptide enhancers with DOPE or gemini surfactants, BM [P/L] and BM [P/G], were also characterized as shown in FIGS. 4(A) and (B). The complexation of pDNA with peptide enhancers and neutral DOPE helper lipids, BM [P/L], had resulted in a major increase in size of the aggregates specially for the particles incorporating greater molar ratios of cationic peptide enhancers (169.0±1.2 vs. 2452.3±332.3 for BM [P_{C}10/L 500] and BM [P_{C}98/L 500], respectively). Further, the BM [P/L] formulations showed low ζ-potentials ranging from −50 mV to +20 mV (e.g., −38.5±0.5 mV, +4.6±1.6 mV, +6.3±0.6 mV, +16.9±0.9 mV and +17.6±2.1 mV for BM [PA308/L 500], BM [P_{B}98/L 500], BM [P_{C}98/L 500], BM [P_{B}98/L 100] and BM [P_{C}98/L 100], respectively). These together suggest the low transfection efficiency and efficacy of the BM [P/L] gene delivery formulations. The physicochemical characterization of BM [P/G] formulations using G7 gemini surfactants (M_{G}=31 μM; ρ=2) demonstrated smaller particle sizes, tighter DNA compactions, and slightly higher ζ-potentials as compared to OBM [G7 31/L 100] formulations (size: 135.9±1.9 nm; ζ-potential: +40.0±0.8 mV for BM [P_{C}98/G7 ³¹]).

Physicochemical characterizations in conjunction with transfection studies (described in detail below) of PDTMG [P/G/L] delivery formulations were investigated for development of potent pDNA delivery systems. It was shown that the incorporation of cationic peptide enhancers (i.e. P_{B-G}; Table 3) for formulating PDTMG systems formed smaller particles with substantially enhanced ζ-potentials as compared to the neutral peptide enhancers (i.e. P_{A}) (Table 5; e.g., size: 159.3±3.2 nm vs. 301.9±17.4 nm; ζ-potential: +49.2±0.9 mV vs. +20.4±0.9 mV for PDTMG-1 [P_{C}49/G7 31/L 100] and PDTMG [P_{A}308/G7 31/L 100], respectively). Through increasing the molar concentrations of cationic peptide enhancers, and fine tuning of gemini surfactants and DOPE/gemini ratios, PDTMG-max was formulated to improve transfection efficiency, efficacy and cell viability (discussed in further detail below). As shown in FIGS. 4(A) and (B), the formulated PDTMG-Max [P_{C}533/G7 17/L 113] formed smaller particles with enhanced ζ-potentials as compared to PDTMG-1 [P_{C}49/G7 31/L 100] (size: 154.3±2.2 nm vs. 195.2±1.6 nm; +56.7±1.0 mV vs. +46.9±0.2 mV for PDTMG-Max [P_{C}533/G7 17/L 113] and PDTMG-1 [P_{C}49/G7 31/L 100], respectively).

Transfection Study and Cell Viability: PDTMG for pDNA Delivery

The transfection efficiency, efficacy and cell viability of various pDNA delivery formulations (Table 6 and Table 7) were investigated in the following 5 categories: BM [G/L], UM [P], BM

[P/L], BM [P/G], PDTMG [P/G/L] by flow cytometry.

The interpretation of flow cytometry data was established to distinguish the transfection efficiency resulting from the cellular uptake of pDNA and the transfection efficacy associated with the mean fluorescence intensity (MFI) of the cells expressing GFP. To understand and interpret flow cytometry data for transfection efficiency and efficacy, the relative measurements were investigated according to control-untreated cells and control-mock pDNA-treated cells with intensity values below 5,000 and 20,000 range, respectively, on the BL1 logarithmic axis (FIG. 10(A)). These taken together, the transfection efficiency of gene delivery formulations was investigated by setting the outlier at 5,000 (TE at low threshold (LT) analysis) for measuring the percentage of pDNA-transfected cells; while, the transfection efficacy was investigated by setting the outlier at either 5,000 (MFI at low threshold (LT) analysis), 10,000 (MFI at high threshold (HT) analysis) and 20,000 (MFI at very high threshold (VHT) analysis) to lower the effect of pDNA-transfected cells with zero GFP expression on the overall mean fluorescence intensity (MFI) of the cells expressing GFP (as illustrated in FIG. 10 in the 2D BL1 vs. RL1 dot plots). In addition, the percentage of cell viability of gene delivery formulations was investigated by setting the outlier at 30,000 on RL1 axis according to the control-untreated-MitoTracker-stained viable cells. This information provides notable insight into the multifactorial considerations for the development of gene delivery systems.

Gemini Surfactants and DOPE Helper Lipid Effects

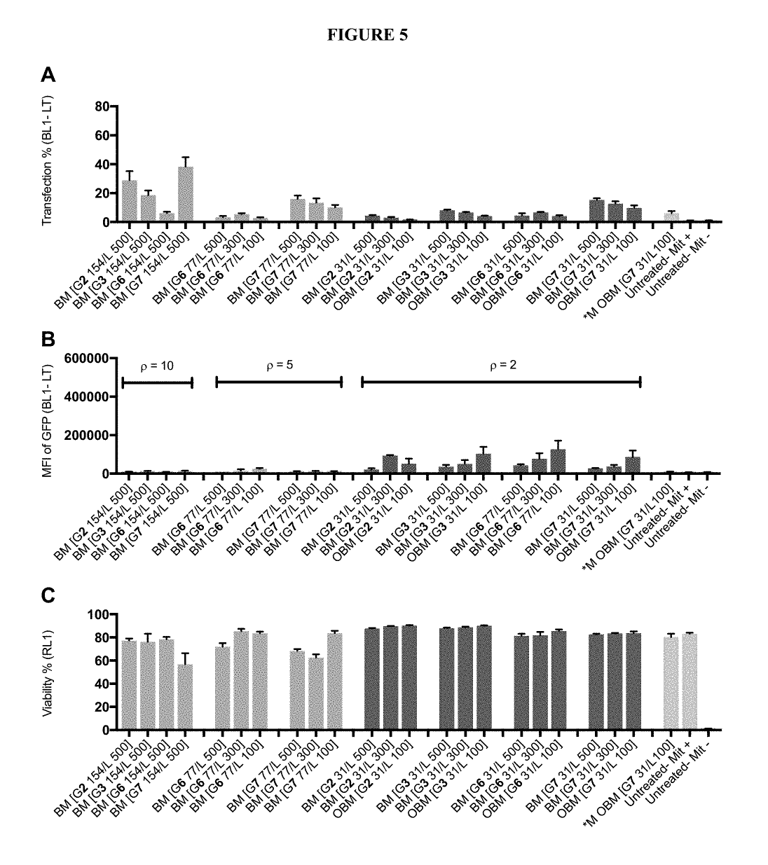

BM [G/L] gene delivery systems were investigated using RGDG-18 (G7; 18-7N(RGDG)), RGDG-12 (G6; 12-7N(RGDG)-12), 18-7NH-18 (G3) and 12-7NH-12 (G2) gemini surfactants and DOPE helper lipids at various ρ values and lipid molarity. The optimization of BM [G/L] delivery systems were carried out by both decreasing the M_{G} from 154 μM (ρ=10), 77 μM (ρ=5), to 31 μM (ρ=2) and M_{L} from 500 μM, 300 μM, to 100 μM. As shown in FIG. 5, the OBM [G 31/L 100] delivery systems resulted in significant improvements in transfection efficacy and cell viability (e.g., MFI: 86,977 vs. 12,937; viability index: 101% vs. 68% for OBM [G7 31/L 100] and BM [G7 154/L 500], respectively). This in contrast, however, resulted in significant reduction in transfection efficiency (e.g., TE: 10% vs. 38% for OBM [G7 31/L 100] and BM [G7 154/L 500], respectively). The challenges with improving the efficiency and efficacy of the BM [G/L] gene delivery systems can be seen where enhancing one either declined or did not significantly improve the other. These results suggest that, in certain conditions, the BM [G/L] delivery systems are less than ideal for pDNA compaction and transfection vs. endosomal escape and pDNA release into the cell cytoplasm and vice versa. For example, the OBM delivery systems using RGDG-18 gemini surfactants, OBM [G7 31/L 100], improved the transfection efficiency without significant changes in transfection efficacy as compared to that of using RGDG-12 or 18-7NH-18 gemini surfactants (TE: 4% and 4%; MFI: 126,954 and 104,881 for OBM [G6 31/L 100] and OBM [G3 31/L 100], respectively).

Non-Covalent Addition of Cationic Peptide Enhancers:

The non-covalent addition of peptide enhancers (i.e., P_{A-G}; Table 3) were investigated for pDNA delivery (i.e., UM [P], BM [P/L], BM [P/G], PDTMG [P/G/L]). As shown in FIG. 6, the addition of cationic peptide enhancers alone, UM [P], or in combination with DOPE helper lipids, BM [P/L], resulted in both reduction in transfection efficiency and efficacy as compared to OBM [G/L] gene delivery systems using G7 gemini surfactants (e.g., TE: 4% vs. 3% vs. 14%; MFI: 14,345 vs. 12,952 vs. 99,566 for UM [P_{C}49], BM [P_{C}49/L 100] and OBM [G7 31/L 100], respectively). While the non-covalent addition of peptide enhancers in combination with G7 gemini surfactants, BM [P/G], could result in comparable or higher transfection efficiency, transfection efficacy was reduced as compared to the OBM [G 31/L 100] gene delivery systems using G7 gemini surfactants (TE: 14% and 20%; MFI: 62957 and 43252 for BM [P_{C}49/G7 31] and BM [P_{C}196/G7 31]).

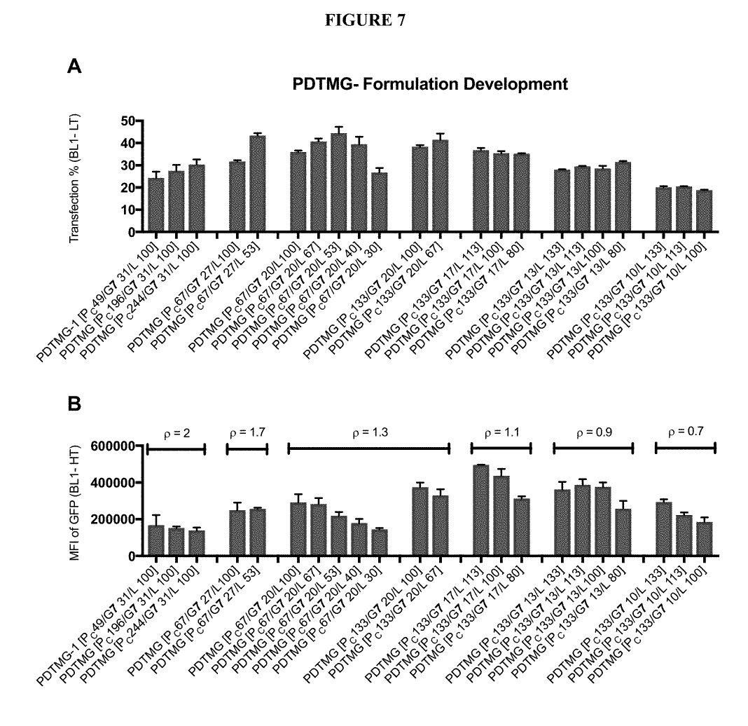

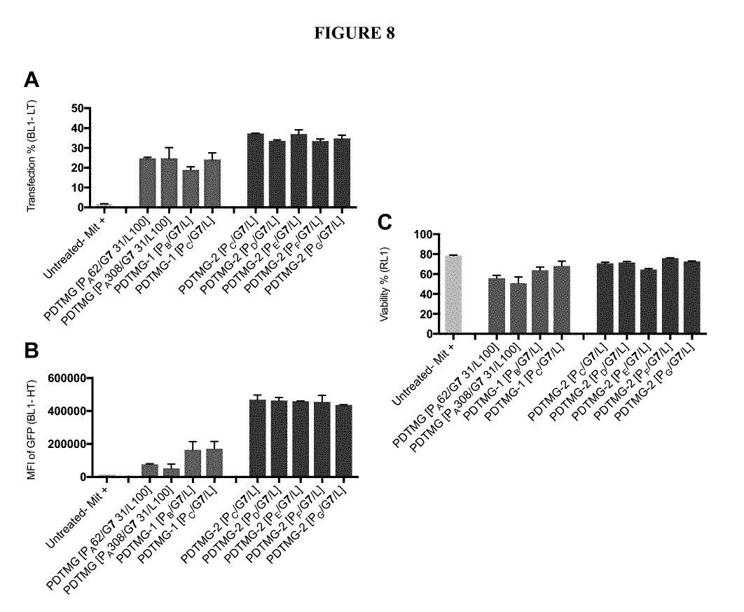

The transfection efficiency, efficacy and cell viability for PDTMG delivery systems were investigated using various peptide enhancers (7 types, P_{A}-P_{G}; Table 3) with different charges (0, 0.5, 3.2, 6.3) and lengths consisting of histidine and/or arginine residues and/or RGD motifs (GRGDSP), using G7 gemini surfactants, and DOPE lipids. The PDTMG delivery systems formulated with P_{C} cationic peptide enhancers resulted in substantial improvements in both transfection efficiency and efficacy as compared to the OBM [G7 31/L 100] gene delivery at the equal lipid molarity (i.e., M_{G} and M_{L}) (FIG. 7; TE: 26% vs. 14%; MFI: 165,805 vs. 99,566 for PDTMG-1 [P_{C}49/G7 31/L 100] and OBM [G7 31/L 100], respectively). As shown in FIG. 8, the PDTMG delivery systems formulated with cationic peptide enhancers (i.e., P_{B}, P_{C}) also resulted in substantial improvements in transfection efficacy and enhanced cell viability with higher or comparable transfection efficiency as compared to the neutral peptide enhancers (i.e., P_{A}) at the equal lipid molarity (i.e., M_{G} and M_{L}) (e.g., TE: 24% vs. 25% vs. 25%; MFI: 171,837 vs. 77,607 vs. 52,843; viability index: 87% vs. 71% vs. 65% for PDTMG-1 [P_{C}49/G7 31/L 100], PDTMG [P_{A}62/G7 31/L 100] and PDTMG [P_{A}308/G7 31/L 100], respectively). Careful formulation studies of the compositional elements was accomplished to advance formulations strategy for PDTMG delivery systems (i.e., PDTMG-1, PDTMG-2, PDTMG-3, PDTMG-Max; Table 4). As shown in FIGS. 6 and 7, the substantial improvement in both transfection efficiency and efficacy of the PDTMG delivery systems was achieved by decreasing gemini surfactants molarity from M_{G}=31 (ρ=2) down to M_{G}=17 (ρ=1.1) while increasing the peptide molarity in the formulation mixtures, and fine tuning of the r values (e.g., TE: 37%; MFI: 544,654 for PDTMG-3 [P_{C}267/G7 17/L 113]). Further, it was shown that regardless of the cationic peptide enhancers used, the transfection efficiency, efficacy and cell viability of a given PDTMG system did not significantly change in these studies (FIG. 8; e.g., TE: 37% and 34%; MFI: 469,464 and 463,465; viability index: 90% and 91% for PDTMG-2 [P_{C}267/G7 17/L 100] and PDTMG-2 [P_{D}267/G7 17/L 100], respectively).

Covalent Functionalization of Gemini Surfactants for High Transfection Efficiency and Efficacy of PDTMG Delivery Systems

Transfection efficiency, efficacy and cell viability of PDTMG [P/G/L] tri-modal delivery systems formulated using 14 different gemini surfactants (G1-G14), P_{C} cationic peptide enhancers and DOPE helper lipids were investigated for development of potent delivery systems.

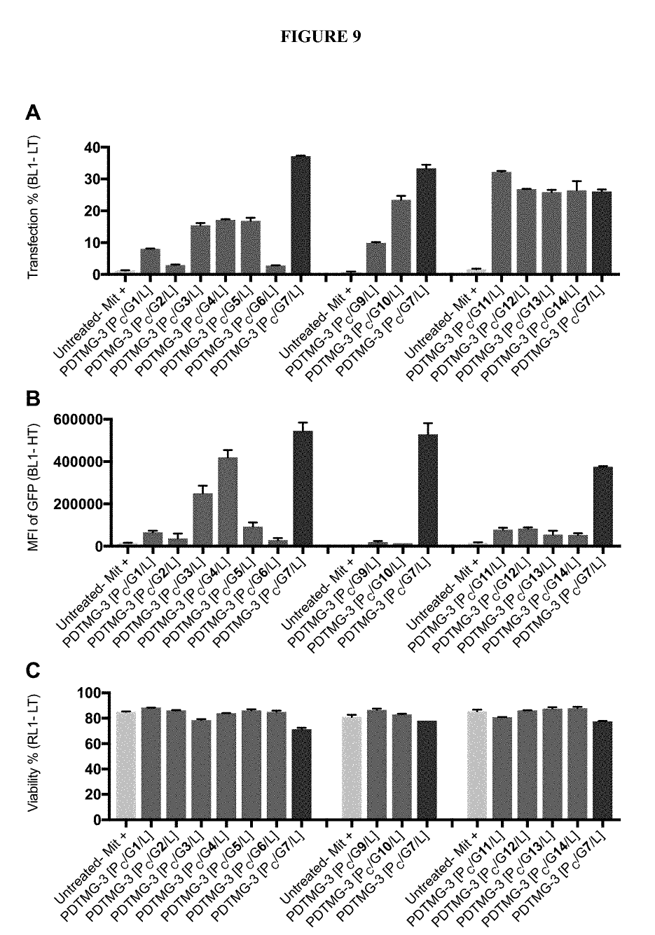

As shown in FIG. 9, both transfection efficiency and efficacy of the PDTMG-3 delivery systems formulated using 18-7NH-18 (G3) gemini surfactants were significantly higher as compared to 18-3-18 (G1), 12-7NH-12 (G2) and RGDG-12 (G6) gemini surfactants (TE: 15% vs. 8% vs. 3% vs. 3%; MFI: 250,524 vs. 66,059 vs. 36,726 vs. 28,841 for PDTMG-3 formulated with [P_{C}/G3/L], [P_{C}/G1/L], [P_{C}/G2/L] and [P_{C}/G6/L], respectively). Further improvements in the transfection efficacy without significant difference in transfection efficiency was achieved for PDTMG-3 formulated using imidazole-functionalized 18 series gemini surfactants (imid-18; G4) (TE: 17%; MFI: 419,498 for PDTMG-3 [Pc/G4/L]). The thiol-functionalization of 18 series gemini surfactants (thiol-18; G5), however, significantly declined the transfection efficacy without changes in transfection efficiency of PDTMG-3 as compared to G4 and G3 gemini surfactants (TE: 17%; MFI: 92,981 for PDTMG-3 [Pc/G5/L]). To further investigate the “proton sponge” effect of the imidazole containing groups, polyhistidine-functionalized gemini surfactants (18-E-PepD; G9) were used in formulating PDTMG-3. It was shown that both transfection efficiency and efficacy were notably reduced for the PDTMG-3 formulated using G9 gemini surfactants as compared to G4 gemini surfactants (TE: 10%; MFI: 18,661 for PDTMG-3 [Pc/G9/L]). While significant improvements in transfection efficiency was achieved for PDTMG-3 formulated using bi-functional polyhistidine-RGD-functionalized 18 series gemini surfactants (18-E-PepE; G10), the transfection efficacy was still noticeably low (TE: 23%; MFI: 14,160 for PDTMG-3 [P_{C}/G10/L]). PDTMG-3 formulated using zwitterionic or cationic arginine rich penta- or hexa-peptide motifs-linked 18 series gemini surfactants (G11 and G12, respectively) showed slight improvements in transfection efficacy of PDTMG-3 without significant changes in transfection efficiency of as compared G9 and G10 gemini surfactants (e.g., TE: 27%; MFI: 83,829 for PDTMG-3 [Pc/G12/L]). The cationic arginine rich hepta-peptides linked 18 series gemini surfactants (G13 and G14), however, resulted in slight decline in transfection efficacy without changes in transfection efficiency of PDTMG-3 as compared to G11 and G12 gemini surfactants (e.g., TE: 26%; MFI: 55,137 for PDTMG-3 [Pc/G13/L]). It was demonstrated that whilst the highest transfection efficiency of PDTMG-3 was achieved by G10-G14 gemini surfactants, the highest transfection efficacy was gained by G4 gemini surfactants in these studies.

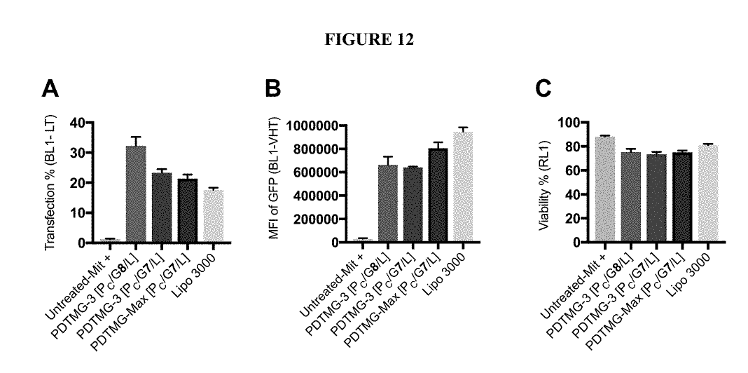

To investigate the effect of short RGD motifs on transfection of PDTMG-3, RGDG and GRGDSPG peptide motifs were conjugated to 18 series gemini surfactants (G7 and G8, respectively). It was revealed that G7 gemini surfactant resulted in tremendous improvements in both transfection efficiency and efficacy of PDTMG-3 delivery systems as compared to gemini surfactants discussed above (G1-G6 and G9-G14) (FIG. 9—e.g., TE: 37%; MFI: 544,654 for PDTMG-3 [Pc/G7/L]). As shown in FIG. 12, G8 gemini surfactants succeeded to significantly enhance the transfection efficiency of PDTMG-3 with slight improvement in the transfection efficacy as compared to G7 gemini surfactants (TE: 32% vs. 23%; MFI: 664,500 vs. 641882 for PDTMG-3 [Pc/G8/L] and PDTMG-3 [Pc/G7/L], respectively). Further advances in formulation strategies, PDTMG-Max were formulated by increasing the P_{C} molarity to M_{P}=533 μM. As shown in FIG. 12, PDTMG-Max formulated using G7 gemini surfactants revealed comparable transfection efficiency and efficacy as compared to the commercially available Lipofectamine™ 3000 reagent (TE: 21% vs. 18%; MFI: 805,854 vs. 946,278 for PDTMG-Max [Pc/G7/L] and Lipofectamine™ 3000, respectively).

Discussion

Transfection efficiency and efficacy of gene delivery formulations in correlation with their physicochemical properties were identified for the development of nucleic acid delivery systems. BM [G/L], OBM [G/L], UM [P], BM [P/L], BM [P/G] and PDTMG [P/G/L]) were formulated using zwitterionic and cationic peptide enhancer (P: P_{A}-P_{G}), gemini surfactants with various spacer groups and alky tails (G: G1-G14; m=12, 18; s=3, 7NH, 7NR₁₋₁₀) and DOPE helper lipids (L) at various ρ and r values at different molarity of the compositional elements in the formulation mixtures (M_{P}, M_{G}, M_{L}).

The physicochemical characterization of the gene delivery formulations by DLS showed that the formulated systems using 18-series gemini surfactants generally formed smaller particles as compared to 12-series gemini surfactants. Transfection study by quantitative flow cytometry demonstrated that while increasing the molar concentrations of 18-series gemini surfactants can improve the transfection efficiency, the transfection efficacy is only functional up to ρ value of 2 (1≤ρ≤2), above which the compaction is detrimental to endosomal release of the plasmid DNA. This value can be potentially increased up to ρ=3 for gemini surfactants with shorter alkyl chains (i.e., 12-series gemini surfactants) (data not shown). It was shown that, rather than the size of the aggregates determining transfection efficiency, the compositional elements comprising the delivery system were shown to play an important role for transfection efficiency. Particle stability of the delivery systems are also important factors for transfection reagents and in vivo applications. As shown in FIG. 5, the OBM [G 31/L 100] delivery systems formulated using 18-series G3 gemini surfactants improved the transfection efficacy by approximately 8 fold while decreased the transfection efficiency by 5 fold as compared to BM [G3 154/L 500] (TE: 4% vs. 19%; WI: 104,381 vs. 12,974, for OBM [G3 31/L 100] and BM [G3 154/L 500, respectively). Using 18-series G7 gemini surfactants-formulated OBM [G 31/L 100] delivery systems increased the transfection efficiency by 2.5 fold but declined the transfection efficacy by 1.2 fold as compared G3 gemini surfactants (TE: 10%; MFI: 86,977 for OBM [G7 31/L 100]). PDTMG delivery systems formulated using P_{A} zwitterionic peptide enhancers, G7 gemini surfactants and DOPE helper lipids improved transfection efficiency to approximately 25% without significant improvements in transfection efficacy (FIG. 8) (TE: 25% and 25%; MFI: 77,607 and 52,843 for PDTMG [P_{A}62/G7 31/L 100] and PDTMG [P_{A}308/G7 31/L 100], respectively). Significant improvements in both transfection efficiency and efficacy was achieved by PDTMG-1,2 delivery systems formulated using cationic peptide enhancers (P_{B}-P_{G}), G7 gemini surfactants and DOPE helper lipids as compared to OBM [G7/L100] (FIGS. 6 and 8) (e.g., TE: 26%; MFI: 165,805 for PDTMG-1 [P_{C}49/G7 31/L100]). As shown in FIGS. 9 and 12, amongst 14 different gemini surfactants [G1-G14], tremendous enhancements in both transfection efficiency and efficacy were demonstrated by PDTMG-3 [P_{C}267/G 17/L 113] delivery systems using G7 and G8 gemini surfactants [e.g., TE: 37%, MFI, 544,654 PDTMG-3 P_{C}267/G7 17/L 113]). Without wishing to be bound by theory, the short RGD peptide motifs (i.e., RGDG, GRGDSPG) linked to 18-series gemini surfactants are believed to provide a reduced steric hindrance for molecular dynamics on the surface of PDTMG-3 nanoparticles and, therefore, it is believed that this exhibited endosomal destabilizing effects in response to cellular environment. This may explain the synergistic effects observed in these experimental studies of the compositional elements of the RGD-18-formulated PDTMG nanocarriers, where it is believed that the 18-series gemini surfactants in conjunction with DOPE helper lipids first stabilized and compacted the cationic peptide enhancers intercalating pDNA at the core of the PDTMG delivery systems, and these together may provide the firm platform for the destabilizing movements of the RGD motifs (R₃, R₄) at the surface of the formulated PDTMG delivery systems for effective endosomal destabilizing functionality in response to the cellular environment; hence, effectively releasing pDNA into the cytoplasm. Without wishing to be bound by theory, it is believed that this phenomenon may be further explained by comparing the formulated PDTMG-3 using the bulky bi-functional polyhistidine-RGD-linked 18 series gemini surfactants or the short RGDG peptide motifs linked to 12-series gemini surfactants, in which both resulted in low transfection efficacy.

Further advances in formulation strategies provided PDTMG-Max, formulated using G7 and G8 gemini surfactants, and by increasing the concentrations of cationic peptide enhancers (M_{P}=533 μM). The considerable amount of cationic peptide enhancers embedded at the core of the nano-sized carriers resulted in amplified endosomal rupture of the delivery system. The formulated PDTMG-Max revealed higher or comparable transfection efficiency and efficacy as compared to the commercially available Lipofectamine™ 3000 reagent.

The results described herein highlight particular trends which may be useful in developing nucleic acid delivery compositions. By way of example, the results described herein suggest that:

[1] while transfection efficiency may be improved by increasing the molar concentrations of gemini surfactants and/or DOPE helper lipids, the transfection efficacy is only functional up to ρ≤3, depending on the DNA compaction associated with the lengths of the alkyl tails of gemini surfactants;

[2] improved transfection efficacy by low dense OMB [G/L] particles formulated at ρ=2 and M_{L}=100, result in low transfection efficiency;

[3] improvements in transfection efficiency of OBM [G/L] particles may be achieved by covalent functionalization of gemini surfactants with zwitterionic or cationic R-functional groups; however, this may result in low transfection efficacy correlated with the DNA compaction;

[4] improvement in transfection efficiency may be achieved by non-covalent addition of zwitterionic peptide enhancers in formulating PDTMG [P/G/L] delivery systems; however, this did not significantly improve transfection efficacy;

[5] non-covalent addition of cationic peptide enhancer embedded at the core of PDTMG delivery systems may both improve transfection efficiency and efficacy; provided that the stable PDTMG particles were formulated with fusogenic R-functionalized gemini surfactants;

[6] zwitterionic R functional moieties with reduced steric hindrance structures (e.g. R₃ and R₄) may be designed and covalently linked to 18-series gemini surfactants (e.g., G7 and G8) to form an active PDTMG nanoparticle. Therefore, the active PDTMG nanoparticles may exhibit endosomal destabilizing effect in response to the cellular environment, and effectively release DNA into the cell cytoplasm.

These studies identify and characterize a wide variety of delivery surfactants and delivery compositions, and factors relevant for developing potent delivery systems and vehicles. By way of example, these studies identify and characterize active PDTMG nanoparticles constructed by careful formulations of the compositional elements comprising [cationic peptide enhancers (such as P_{B}-P_{G}), short RGD peptide motifs (RGDG, GRGDSPG)-linked 18 series gemini surfactants (such as G7 and G8), and DOPE helper lipids]. The PDTMG delivery systems are identified as an important platform for designing and developing targeted delivery of nucleic acids to cells. By way of example, such nucleic acids may include, but are not limited to, nucleotide-based therapeutics (i.e., pDNA, shRNA plasmid, siRNA, etc.) to be delivered to specific cell lines. Results provided herein suggest that, in certain embodiments, delivery compositions described herein may be applicable to in vivo nucleotide-based gene therapy and/or DNA vaccine applications, for example.

One or more illustrative embodiments have been described by way of example. It will be understood to persons skilled in the art that a number of variations and modifications can be made without departing from the scope of the invention as defined in the claims.

TABLE 1
Fourteen gemini surfactants (m-s-m formula) studied in this report. m = 12 and 18
carbon alkyl chains, s = 3 (3 methylene unit), 7NH (imino-substituted-7 methylene unit), 7NR
(R-linked-imino-substituted-7-methylene unit) spacer groups. R = R₁-R₁₀ functional moieties

| Compound | | | |
|---|---|---|---|
| # | Names | Compound formula | |
| G1 | 18-3-18 | m-3-m | 18-3-18 |
| G2 | 12-7NH-12 | m-7NH-m | 12-7NH-12 |
| G3 | 18-7NH-18 | | 18-7NH-18 |
| G4 | Imid-18 | m-7NR-m | 18-7NR₁-18 (R₁ = imidazolpropionyl) |
| G5 | Thiol-18 | | 18-7NR₂-18 (R₂ = thiopropionyl) |
| G6 | RGDG-12 | | 12-7NR₃-12 (R₃ = RGDG—) |
| G7 | RGDG-18 | | 18-7NR₃-18 (R₃ = RGDG—) |
| G8 | GRGDSPG-18 | | 18-7NR₄-18 (R₄ = GRGDSPG—) |
| G9 | 18-E-PepD | | 18-7NR₅-18 (R₅ = —E(H)₅) |
| G10 | 18-E-PepE | | 18-7NR₆-18 (R₆ = —EGRGDSPG(H)₅) |
| G11 | 18-Suc-E₂GR₂ | | 18-7NR₇-18 (R₇ = -Suc-(E)₂G(R)₂) |
| G12 | 18-Suc-E₂GR₃ | | 18-7NR₈-18 (R₈ = -Suc-(E)₂G(R)₃) |
| G13 | 18-Suc-E₂G₃R₃ | | 18-7NR₉-18 (R₉ = -Suc-(E)₂(G)₃(R)₃) |
| G14 | 18-Suc-DEG₃R₃ | | 18-7NR₁₀-18 (R₁₀ = -Suc-DE(G)₃(R)₃) |

TABLE 2
Characterization of m-7NR-m gemini surfactants (m = 12, 18; R = R₁-R₁₀). The identity
of the synthesized G4-G14 m-7NR-m gemini surfactants were confirmed by ESI-MS and the
purifications were conducted by RP-HPLC with a linear gradient of solvent B on 300SB-C18
semi-preparative column; mobile phases: solvent A (water/TFA: 99.9/0.1, v/v) and solvent B
(MeCN/TFA: 99.9/0.1, v/v); flow rate: 10 mL/min; UV detection: 214 nm.

| Compounds | | MW | | |
|---|---|---|---|---|
| # | Names | (g/mol) | ESI-MS (m/z) | RP-HPLC |
| G4 | Imid-18 | 816.42 | 407.92 [M] ²⁺, 272.28 [M] ³⁺ | 60-100% B in 15 min |
| G5 | Thiol-18 | 782.43 | 390.90 [M] ²⁺ | 60%-100% B in 15 min |
| G6 | RGDG-12 | 911.36 | 303.92 [M] ³⁺ | 40%-60% B in 15 min |
| G7 | RGDG-18 | 1079.67 | 359.99 [M] ³⁺ | 60%-100% B in 15 min |
| G8 | GRGDSPG-18 | 1320.92 | 660.03 [M] ²⁺, 440.35 [M] ³⁺, 330.52 [M] ⁴⁺ | 60%-100% B in 15 min |
| G9 | 18-E-PepD | 1509.11 | 754.06 [M] ²⁺, 503.04 [M] ³⁺, 377.53 [M] ⁴⁺ | 50%-100% B in 10 min |
| G10 | 18-E-PepE | 2135.73 | 1067.69 [M] ²⁺, 712.13 [M] ³⁺, 534.35 [M] ⁴⁺ | 50%-100% B in 20 min |
| | | | 427.68 [M] ⁵⁺, 356.57 [M] ⁶⁺ | |
| G11 | 18-Suc-E₂GR₂ | 1421.04 | 710.06 [M] ²⁺, 473.71 [M] ³⁺, 355.53 [M] ⁴⁺ | 50%-100% B in 20 min |
| G12 | 18-Suc-E₂GR₃ | 1577.22 | 525.74 [M] ³⁺, 394.56 [M] ⁴⁺ | 50%-100% B in 20 min |
| G13 | 18-Suc-E₂G₃R₃ | 1691.32 | 564.09 [M] ³⁺, 423.32 [M] ⁴⁺ | 50%-100% B in 20 min |
| G14 | 18-Suc-DEG₃R₃ | 1679.31 | 559.42 [M] ³⁺, 419.82 [M] ⁴⁺ | 50%-100% B in 20 min |

TABLE 3
Amino acid sequence, net charge (at pH = 7)
and molecular weight of seven peptide enhancers
(P_{A}-P_{G}) with different lengths consisting of histidine and/or
arginine residues and/or RGD (GRGDSP) motifs.

| Peptide | Sequence (N- | Net charge | MW |
|---|---|---|---|
| enhancers | to C-terminus) | at pH 7 | (g/mol) |
| P_{A} (7a.a.) | GRGDSPG | 0 | 664.63 |
| P_{B} (10a.a.) | H(R)₃H(R)₃HG | 6.3 | 1423.6 |
| P_{C} (17a.a.) | GRGDSPGH(R)₃H(R)₃HG | 6.3 | 2052.22 |
| P_{D} (5a.a.) | (H)₅ | 0.5 | 703.71 |
| P_{E} (12a.a.) | GRGDSPG(H)₅ | 0.5 | 1330.33 |
| P_{F} (15a.a.) | (H)₂R(H)₇R(H)₃G | 3.2 | 2033.11 |
| P_{G} (22a.a.) | GRGDSPG(H)₂R(H)₇R(H)₃G | 3.2 | 2659.7 |

TABLE 4
Selected gene delivery systems formulated using peptide enhancers (P) and/or gemini
surfactants (G) and/or DOPE helper lipids (L). (A) Detailed information on formulating Uni-
Modal (UM [P M_{P}]), Bi-Modal (BM [G M_{G}/L M_{L}], BM [P M_{P}/L M_{L}], BM [P M_{P}/G M_{G}]) and
Tri-Modal (PDTMG [P M_{P}/G M_{G}/L M_{L}]) delivery systems containing 0.5 μg (500 ng) DNA
(50 μL transfection reagent per well of a 24 well plate). (B) Scaling methods of transfection
reagents (10 μL, 50 μL, 250 μL) for formulating 100 ng, 500 ng and 2500 ng DNA used per well
of 96-well, 24-well and 6-well plates, respectively.

| A) Formulations containing | Peptide (P) | | Gemini (G) | | DOPE (L) | | | |
|---|---|---|---|---|---|---|---|---|
| 0.5 μg (0.77 nmol) pDNA per | n_{P} | M_{P} | n_{G} | M_{G} | n_{L} | M_{L} | | |
| well of a 24-well plate | (nmol) | (μM) | (nmol) | (μM) | (nmol) | (μM) | *ρ | r |
| BM [G 154/L 500] | 0.0 | 0 | 7.69 | 154 | 25.00 | 500 | 10 | 3.3 |
| BM [G 31/L 500] | 0.0 | 0 | 1.54 | 31 | 25.00 | 500 | 2 | 16.1 |
| OBM [G 31/L 100] | 0.0 | 0 | 1.54 | 31 | 5.00 | 100 | 2 | 3.3 |
| UM [P 49] | 2.4 | 49 | 0.00 | 0 | 0.00 | 0 | — | — |
| BM [P 49/L 100] | 2.4 | 49 | 0.00 | 0 | 5.00 | 100 | — | — |
| BM [P 49/G 31] | 2.4 | 49 | 1.54 | 31 | 0.00 | 0 | 2 | — |
| PDTMG-1 [P 49/G 31/L 100] | 2.4 | 49 | 1.54 | 31 | 5.00 | 100 | 2 | 3.3 |
| PDTMG-2 [P 267/G 17/L 100] | 13.3 | 267 | 0.8 | 17 | 5.00 | 100 | 1.1 | 6.0 |
| PDTMG-3 [P 267/G 17/L 113] | 13.3 | 267 | 0.8 | 17 | 5.67 | 113 | 1.1 | 6.8 |
| PDTMG-Max [P 533/G 17/L 113] | 26.7 | 533 | 0.8 | 17 | 5.67 | 113 | 11 | 6.8 |

| B) Formulation preparations | 96-well plate | 24-well plate | 6-well plate |
|---|---|---|---|
| DNA per well | 100 ng | 500 ng | 2500 ng |
| Transfection reagent per well | 10 μL | 50 μL | 250 μL |

*ρ is calculated according to di-cationic gemini surfactants.

TABLE 5
Hydrodynamic diameter, PDI and ζ-potential of selected formulations consisting of
peptide enhancers (P_{A}-P_{C}) and/or m-7NR-m gemini surfactants (G6, G7) and/or DOPE helper
lipids (L). Data points are presented as mean ± SD, n = 3.

| Formulations | Average Size (d.nm) | PDI | ζ-potential (mV) |
|---|---|---|---|
| BM [G7 154/L 500] | 291.5 ± 20.8 | 0.339 ± 0.010 | +63.4 ± 1.3 |
| BM [G7 31/L 500] | 3738.7 ± 172.4 | 0.681 ± 0.230 | −14.1 ± 0.5 |
| OBM [G7 31/L 100] | 289.4 ± 8.8 | 0.475 ± 0.006 | +38.3 ± 0.5 |
| UM [G7 31] | 408.1 ± 6.8 | 0.517 ± 0.038 | +34.0 ± 0.5 |
| BM [G6 154/L 500] | 517.5 ± 49.0 | 0.516 ± 0.059 | +39.0 ± 2.1 |
| BM [G6 31/L 500] | 1483.7 ± 157.7 | 0.886 ± 0.041 | −5.6 ± 0.2 |
| OBM [G6 31/L 100] | 477.1 ± 16.2 | 0.447 ± 0.022 | +21.9 ± 1.6 |
| UM [G6 31] | 1925.3 ± 271.4 | 0.423 ± 0.098 | +4.2 ± 3.1 |
| UM [P_{A}62] | 147.2 ± 1.4 | 0.498 ± 0.012 | −1.2 ± 0.2 |
| UM [P_{A}308] | 119.2 ± 7.0 | 0.522 ± 0.065 | −2.2 ± 0.2 |
| UM [P_{B}10] | 297.4 ± 18.8 | 0.368 ± 0.044 | −1.1 ± 0.3 |
| UM [P_{B}49] | 230.4 ± 3.2 | 0.332 ± 0.024 | +21 ± 0.6 |
| UM [P_{B}98] | 192.9 ± 0.5 | 0.401 ± 0.003 | +24.1 ± 0.5 |
| UM [P_{C}10] | 209.4 ± 1.9 | 0.249 ± 0.006 | +2.2 ± 0.2 |
| UM [P_{C}49] | 216.8 ± 0.6 | 0.293 ± 0.022 | +19.8 ± 0.2 |
| UM [P_{C}98] | 123.6 ± 1.2 | 0.366 ± 0.010 | +20.0 ± 1.4 |
| BM [P_{A}62/L 500] | 111.2 ± 0.4 | 0.233 ± 0.002 | −46.9 ± 1.6 |
| BM [P_{A}308/L 500] | 113.4 ± 0.7 | 0.236 ± 0.008 | −38.5 ± 0.5 |
| BM [P_{B}10/L 500] | 139.6 ± 1.0 | 0.232 ± 0.002 | −24.8 ± 0.6 |
| BM [P_{B}49/L 500] | 2741.7 ± 79.7 | 0.308 ± 0.139 | +2.4 ± 0.4 |
| BM [P_{B}98/L 500] | 3904.3 ± 219.3 | 1 ± 0.000 | +4.6 ± 1.6 |
| BM [P_{C}10/L 500] | 169.0 ± 1.2 | 0.181 ± 0.002 | −8.0 ± 0.1 |
| BM [P_{C}49/L 500] | 4154.3 ± 471.6 | 0.67 ± 0.285 | +7.6 ± 2.4 |
| BM [P_{C}98/L 500] | 2452.3 ± 332.3 | 0.321 ± 0.086 | +6.3 ± 0.6 |
| BM [P_{B}98/L 100] | 261.1 ± 7.1 | 0.316 ± 0.026 | +16.7 ± 0.9 |
| BM [P_{C}98/L 100] | 341.9 ± 12.5 | 0.405 ± 0.020 | +17.6 ± 2.1 |
| BM [P_{B}49/G7 31] | 186.7 ± 2.4 | 0.249 ± 0.007 | +44.7 ± 0.6 |
| BM [P_{B}98/G7 31] | 150.6 ± 1.0 | 0.220 ± 0.003 | +48.5 ± 0.4 |
| BM [P_{C}49/G7 31] | 226.4 ± 6.2 | 0.276 ± 0.023 | +47.7 ± 0.8 |
| BM [P_{C}98/G7 31] | 135.9 ± 1.9 | 0.284 ± 0.012 | +40.0 ± 0.8 |
| PDTMG [P_{A}308/G7 31/L 100] | 301.9 ± 17.4 | 0.447 ± 0.002 | +20.4 ± 0.9 |
| PDTMG-1 [P_{B}49/G7 31/L 100] | 192.3 ± 0.7 | 0.137 ± 0.015 | +45.4 ± 0.6 |
| PDTMG [P_{B}98/G7 31/L 100] | 159.3 ± 3.2 | 0.152 ± 0.032 | +49.6 ± 0.9 |
| PDTMG-1 [P_{C}49/G7 31/L 100] | 195.2 ± 1.6 | 0.174 ± 0.017 | +46.9 ± 0.2 |
| PDTMG [P_{C}98/G7 31/L 100] | 158.8 ± 3.5 | 0.144 ± 0.058 | +51.5 ± 0.8 |
| PDTMG-3 [P_{C}267/G7 17/L 113] | 168.6 ± 0.6 | 0.188 ± 0.029 | +51.1 ± 0.3 |
| PDTMG-Max [P_{C}533/G7 17/L 113] | 154.3 ± 2.2 | 0.144 ± 0.019 | +56.7 ± 1.0 |

TABLE 6
Details on formulating Uni-Modal (UM [P M_{P}]), Bi-Modal (BM [G M_{G}/L M_{L}],
BM [P M_{P}/L M_{L}], BM [P M_{P}/G M_{G}]) delivery systems
at various molar concentration of compositional elements.

| | | *n_{P} | *n_{G} | *n_{L} | M_{P} | M_{G} | M_{L} | |
|---|---|---|---|---|---|---|---|---|
| # | Formulations | (nmol) | (nmol) | (nmol) | (μM) | (μM) | (μM) | r |
| F1 | BM [G 154/L 500] | 0.0 | 7.69 | 25.00 | 0 | 154 | 500 | 3.3 |
| F2 | BM [G 154/L300] | 0.0 | 7.69 | 15.00 | 0 | 154 | 300 | 2.0 |
| F3 | BM [G 154/L100] | 0.0 | 7.69 | 5.00 | 0 | 154 | 100 | 0.7 |
| F4 | BM [G 77/L500] | 0.0 | 3.85 | 25.00 | 0 | 77 | 500 | 6.5 |
| F5 | BM [G 77/L300] | 0.0 | 3.85 | 15.00 | 0 | 77 | 300 | 3.9 |
| F6 | BM [G 77/L100] | 0.0 | 3.85 | 5.00 | 0 | 77 | 100 | 1.3 |
| F7 | BM [G 31/L 500] | 0.0 | 1.54 | 25.00 | 0 | 31 | 500 | 16.2 |
| F8 | BM [G 31/L300] | 0.0 | 1.54 | 15.00 | 0 | 31 | 300 | 9.7 |
| F9 | OBM [G 31/L 100] | 0.0 | 1.54 | 5.00 | 0 | 31 | 100 | 3.3 |
| F10 | UM [G 31] | 0.0 | 1.54 | 0.00 | 0 | 31 | 0 | — |
| F11 | UM [P 10] | 0.5 | 0.00 | 0.00 | 10 | 0 | 0 | — |
| F12 | UM [P 49] | 2.4 | 0.00 | 0.00 | 49 | 0 | 0 | — |
| F13 | UM [P 62] | 3.1 | 0.00 | 0.00 | 62 | 0 | 0 | — |
| F14 | UM [P 98] | 4.9 | 0.00 | 0.00 | 98 | 0 | 0 | — |
| F15 | UM [P 196] | 9.8 | 0.00 | 0.00 | 196 | 0 | 0 | — |
| F16 | UM [P 308] | 15.4 | 0.00 | 0.00 | 308 | 0 | 0 | — |
| F17 | BM [P 10/L 500] | 0.5 | 0.00 | 25.00 | 10 | 0 | 500 | — |
| F18 | BM [P 49/L 500] | 2.4 | 0.00 | 25.00 | 49 | 0 | 500 | — |
| F19 | BM [P 62/L 500] | 3.1 | 0.00 | 25.00 | 62 | 0 | 500 | — |
| F20 | BM [P 98/L 500] | 4.9 | 0.00 | 25.00 | 98 | 0 | 500 | — |
| F21 | BM [P 308/L 500] | 15.4 | 0.00 | 25.00 | 308 | 0 | 500 | — |
| F22 | BM [P 49/L 100] | 2.4 | 0.00 | 5.00 | 49 | 0 | 100 | — |
| F23 | BM [P 98/L 100] | 4.9 | 0.00 | 5.00 | 98 | 0 | 100 | — |
| F24 | BM [P 49/G 31] | 2.4 | 1.54 | 0.00 | 49 | 31 | 0 | — |
| F25 | BM [P 98/G 31] | 4.9 | 1.54 | 0.00 | 98 | 31 | 0 | — |
| F26 | BM [P 196/G 31] | 9.8 | 1.54 | 0.00 | 196 | 31 | 0 | — |

TABLE 7
Details on formulating Tri-Modal (PDTMG [P M_{P}/G M_{G}/L M_{L}]) delivery systems at
various molar concentration of compositional elements.

| | | *n_{P} | *n_{G} | *n_{L} | M_{P} | M_{G} | M_{L} | |
|---|---|---|---|---|---|---|---|---|
| # | Formulations | (nmol) | (nmol) | (nmol) | (μM) | (μM) | (μM) | r |
| F27 | PDTMG-1 [P 49/G 31/L 100] | 2.4 | 1.54 | 5.00 | 49 | 31 | 100 | 3.3 |
| F28 | PDTMG [P 62/G 31/L 100] | 3.1 | 1.54 | 5.00 | 62 | 31 | 100 | 3.3 |
| F29 | PDTMG [P 98/G 31/L 100] | 4.9 | 1.54 | 5.00 | 98 | 31 | 100 | 3.3 |
| F30 | PDTMG [P 196/G 31/L 100] | 9.8 | 1.54 | 5.00 | 196 | 31 | 100 | 3.3 |
| F31 | PDTMG [P 244/G 31/L 100] | 12.2 | 1.54 | 5.00 | 244 | 31 | 100 | 3.3 |
| F32 | PDTMG [P 308/G 31/L 100] | 15.4 | 1.54 | 5.00 | 308 | 31 | 100 | 3.3 |
| F33 | PDTMG [P 67/G 27/L 100] | 3.3 | 1.3 | 5.00 | 67 | 27 | 100 | 3.8 |
| F34 | PDTMG [P 67/G 27/L 53] | 3.3 | 1.3 | 2.67 | 67 | 27 | 53 | 2.0 |
| F35 | PDTMG [P 67/G 20/L 100] | 3.3 | 1.0 | 5.00 | 67 | 20 | 100 | 5.0 |
| F36 | PDTMG [P 67/G 20/L 67] | 3.3 | 1.0 | 3.33 | 67 | 20 | 67 | 3.3 |
| F37 | PDTMG [P 67/G 20/L 53] | 3.3 | 1.0 | 2.67 | 67 | 20 | 53 | 2.7 |
| F38 | PDTMG [P 67/G 20/L 40] | 3.3 | 1.0 | 2.00 | 67 | 20 | 40 | 2.0 |
| F39 | PDTMG [P 67/G 20/L 30] | 3.3 | 1.0 | 1.50 | 67 | 20 | 30 | 1.5 |
| F40 | PDTMG [P 133/G 20/L 100] | 6.7 | 1.0 | 5.00 | 133 | 20 | 100 | 5.0 |
| F41 | PDTMG [P 133/G 20/L 67] | 6.7 | 1.0 | 3.33 | 133 | 20 | 67 | 3.3 |
| F42 | PDTMG [P 133/G 17/L 113] | 6.7 | 0.8 | 5.67 | 133 | 17 | 113 | 6.8 |
| F43 | PDTMG [P 133/G 17/L 100] | 6.7 | 0.8 | 5.00 | 133 | 17 | 100 | 6.0 |
| F44 | PDTMG [P 133/G 17/L 80] | 6.7 | 0.8 | 4.00 | 133 | 17 | 80 | 4.8 |
| F45 | PDTMG-3 [P 267/G 17/L113] | 13.3 | 0.8 | 5.67 | 267 | 17 | 113 | 6.8 |
| F46 | PDTMG-2 [P 267/G 17/L100] | 13.3 | 0.8 | 5.00 | 267 | 17 | 100 | 6.0 |
| F47 | PDTMG-Max [P 533/G 17/L 113] | 26.7 | 0.8 | 5.67 | 533 | 17 | 113 | 6.8 |
| F48 | PDTMG [P 133/G 13/L 133] | 6.7 | 0.7 | 6.67 | 133 | 13 | 133 | 10.0 |
| F49 | PDTMG [P 133/G 13/L 113] | 6.7 | 0.7 | 5.67 | 133 | 13 | 113 | 8.5 |
| F50 | PDTMG [P 133/G 13/L 100] | 6.7 | 0.7 | 5.00 | 133 | 13 | 100 | 7.5 |
| F51 | PDTMG [P 133/G 13/L 80] | 6.7 | 0.7 | 4.00 | 133 | 13 | 80 | 6.0 |
| F52 | PDTMG [P 133/G 10/L 133] | 6.7 | 0.5 | 6.67 | 133 | 10 | 133 | 13.3 |
| F53 | PDTMG [P 133/G 10/L 113] | 6.7 | 0.5 | 5.67 | 133 | 10 | 113 | 11.3 |
| F54 | PDTMG [P 133/G 10/L 100] | 6.7 | 0.5 | 5.00 | 133 | 10 | 100 | 10.0 |

### REFERENCES

1. Dunehoo, A. L., et al., Cell adhesion molecules for targeted drug delivery. Journal of pharmaceutical sciences, 2006. 95(9): p. 1856-1872.
2. Mintzer, M. A. and E. E. Simanek, Nonviral vectors for gene delivery. Chemical reviews, 2008. 109(2): p. 259-302.
3. Pack, D. W., D. Putnam, and R. Langer, Design of imidazole-containing endosomolytic biopolymers for gene delivery. Biotechnology and Bioengineering, 2000. 67(2): p. 217-223.
4. Midoux, P., et al., Chemical vectors for gene delivery: a current review on polymers, peptides and lipids containing histidine or imidazole as nucleic acids carriers. British journal of pharmacology, 2009. 157(2): p. 166-178.
5. Lin, C., et al., Random and block copolymers of bioreducible poly (amido amine) s with high-and low-basicity amino groups: study of DNA condensation and buffer capacity on gene transfection. Journal of Controlled Release, 2007. 123(1): p. 67-75.
6. Koren, E. and V. P. Torchilin, Cell-penetrating peptides: breaking through to the other side. Trends in molecular medicine, 2012. 18(7): p. 385-393.
7. Lo, S. L. and S. Wang, An endosomolytic Tat peptide produced by incorporation of histidine and cysteine residues as a nonviral vector for DNA transfection. Biomaterials, 2008. 29(15): p. 2408-2414.
8. Menger, F. M. and C. Littau, Gemini-surfactants: synthesis and properties. Journal of the American Chemical Society, 1991. 113(4): p. 1451-1452.
9. Zana, R., M. Benrraou, and R. Rueff, Alkanediyl-. alpha.,. omega.-bis (dimethylalkylammonium bromide) surfactants. 1. Effect of the spacer chain length on the critical micelle concentration and micelle ionization degree. Langmuir, 1991. 7(6): p. 1072-1075.
10. Menger, F. and C. Littau, Gemini surfactants: a new class of self-assembling molecules. Journal of the American Chemical Society, 1993. 115(22): p. 10083-10090.
11. Zana, R. and Y. Talmon, Dependence of aggregate morphology on structure of dimeric surfactants. Nature, 1993. 362(6417): p. 228-230.
12. Rosen, M. J. and L. D. Song, Dynamic surface tension of aqueous surfactant solutions 8. Effect of spacer on dynamic properties of gemini surfactant solutions. Journal of colloid and interface science, 1996. 179(1): p. 261-268.
13. Wettig, S. and R. Verrall, Thermodynamic studies of aqueous m-s-m gemini surfactant systems. Journal of colloid and interface science, 2001. 235(2): p. 310-316.
14. Wettig, S. D., et al., Thermodynamic and aggregation properties of aza-and imino-substituted gemini surfactants designed for gene delivery. Physical Chemistry Chemical Physics, 2007. 9(7): p. 871-877.
15. Wettig, S. D., R. E. Verrall, and M. Foldvari, Gemini surfactants: a new family of building blocks for non-viral gene delivery systems. Current gene therapy, 2008. 8(1): p. 9-23.
16. Donkuru, M., et al., Designing pH-sensitive gemini nanoparticles for non-viral gene delivery into keratinocytes. Journal of Materials Chemistry, 2012. 22(13): p. 6232-6244.
17. Sharma, V. D. and M. A. Ilies, Heterocyclic Cationic Gemini Surfactants: A Comparative Overview of Their Synthesis, Self-assembling, Physicochemical, and Biological Properties. Medicinal research reviews, 2014. 34(1): p. 1-44.
18. Felgner, P. L., et al., Lipofection: a highly efficient, lipid-mediated DNA-transfection procedure. Proceedings of the National Academy of Sciences, 1987. 84(21): p. 7413-7417.
19. Felgner, J. H., et al., Enhanced gene delivery and mechanism studies with a novel series of cationic lipid formulations. Journal of Biological Chemistry, 1994. 269(4): p. 2550-2561.
20. Kirby, A. J., et al., Gemini surfactants: new synthetic vectors for gene transfection. Angewandte Chemie International Edition, 2003. 42(13): p. 1448-1457.
21. Zuhorn, I. S., et al., Nonbilayer phase of lipoplex-membrane mixture determines endosomal escape of genetic cargo and transfection efficiency. Molecular therapy, 2005. 11(5): p. 801-810.
22. Ewert, K., et al., Cationic lipid-DNA complexes for gene therapy: understanding the relationship between complex structure and gene delivery pathways at the molecular level. Current medicinal chemistry, 2004. 11(2): p. 133-149.
23. Menger, F. M. and J. S. Keiper, Gemini surfactants. Angewandte Chemie International Edition, 2000. 39(11): p. 1906-1920.
24. Majzoub, R. N., et al., Uptake and transfection efficiency of PEGylated cationic liposome—DNA complexes with and without RGD-tagging. Biomaterials, 2014. 35(18): p. 4996-5005.
25. Mohammed-Saeid, W., et al., Design and Evaluation of RGD-Modified Gemini Surfactant-Based Lipoplexes for Targeted Gene Therapy in Melanoma Model. Pharmaceutical Research, 2017: p. 1-11.
26. Krasnykh, V., et al., Characterization of an adenovirus vector containing a heterologous peptide epitope in the HI loop of the fiber knob. Journal of virology, 1998. 72(3): p. 1844-1852.
27. Dmitriev, I., et al., An adenovirus vector with genetically modified fibers demonstrates expanded tropism via utilization of a coxsackievirus and adenovirus receptor-independent cell entry mechanism. Journal of virology, 1998. 72(12): p. 9706-9713.
28. Alqawlaq, S., et al., Preclinical development and ocular biodistribution of gemini-DNA nanoparticles after intravitreal and topical administration: towards non-invasive glaucoma gene therapy. Nanomedicine: Nanotechnology, Biology and Medicine, 2014. 10(8): p. 1637-1647.
29. Gharagozloo, M., et al., A flow cytometric approach to study the mechanism of gene delivery to cells by gemini-lipid nanoparticles: an implication for cell membrane nanoporation. Journal of nanobiotechnology, 2015. 13(1): p. 62.

All references cited herein and elsewhere in the present specification are hereby incorporated by reference in their entireties.

SEQUENCE LISTING

SEQ ID NO: 1 (Artificial Sequence; P_{A}):
GRGDSPG
SEQ ID NO: 2 (Artificial Sequence; P_{B}):
HRRRHRRRHG
SEQ ID NO: 3 (Artificial Sequence; P_{C}):
GRGDSPGHRRRHRRRHG
SEQ ID NO: 4 (Artificial Sequence; P_{D}):
HHHHH
SEQ ID NO: 5 (Artificial Sequence; P_{E}):
GRGDSPGHHHHH
SEQ ID NO: 6 (Artificial Sequence; P_{F}):
HHRHHHHHHHRHHHG
SEQ ID NO: 7 (Artificial Sequence; P_{G}):
GRGDSPGHHRHHHHHHHRHHHG
SEQ ID NO: 8 (Artificial Sequence; R₃):
RGDG
SEQ ID NO: 9 (Artificial Sequence; R₄):
GRGDSPG
SEQ ID NO: 10 (Artificial Sequence; R₆):
EGRGDSPGHHHHH
SEQ ID NO: 11 (Artificial Sequence; R₅):
EHHHHH
SEQ ID NO: 12 (Artificial Sequence;
R₇ (Suc not shown)):
EEGRR
SEQ ID NO: 13 (Artificial Sequence;
R8 (Suc not shown)):
EEGRRR
SEQ ID NO: 14 (Artificial Sequence;
R₉ (Suc not shown)):
EEGGGRRR
SEQ ID NO: 15 (Artificial Sequence;
R₁₀ (Suc not shown)):
DEGGGRRR
SEQ ID NO: 16 (Artificial Sequence;
GRGDSP Motif):
GRGDSP

1. A tri-modal nucleic acid delivery composition comprising:
at least one peptide enhancer;
at least one surfactant; and
at least one helper lipid.

2. The tri-modal nucleic acid delivery composition according to claim 1, wherein the peptide enhancer is zwitterionic, cationic, and/or comprises at least one histidine, lysine, or arginine residue.

3. The tri-modal nucleic acid delivery composition according to claim 1, wherein the peptide enhancer comprises an RGD sequence motif.

4. The tri-modal nucleic acid delivery composition according to claim 1, wherein the peptide enhancer comprises an amino acid sequence of P_{A} (GRGDSPG; SEQ ID NO: 1), P_{B} (H(R)₃H(R)₃HG; SEQ ID NO: 2), P_{C} (GRGDSPGH(R)₃H(R)₃HG; SEQ ID NO: 3), P_{D} ((H)₅; SEQ ID NO: 4), P_{E} (GRGDSPG(H)₅; SEQ ID NO: 5), P_{F} ((H)₂R(H)₇R(H)₃G; SEQ ID NO: 6), P_{G} (GRGDSPG(H)₂R(H)₇R(H)₃G; SEQ ID NO: 7), or GRGDSP (SEQ ID NO: 16).

5. (canceled)

6. The tri-modal nucleic acid delivery composition according to claim 1, wherein the surfactant comprises a cationic gemini surfactant

7.-13. (canceled)

14. The tri-modal nucleic acid delivery composition of claim 1, wherein the surfactant is functionalized with a functional moiety which comprises an imidazole-containing functional group, a thiol-containing functional group, a linear RGD-containing peptide functional group, a polyhistidine-containing peptide functional group, a bifunctional RGD-polyhistidine-containing peptide functional group, a zwitterionic and/or cationic arginine-rich peptide functional group, or any combination thereof.

15. The tri-modal nucleic acid delivery composition of claim 14, wherein the functional moiety comprises:

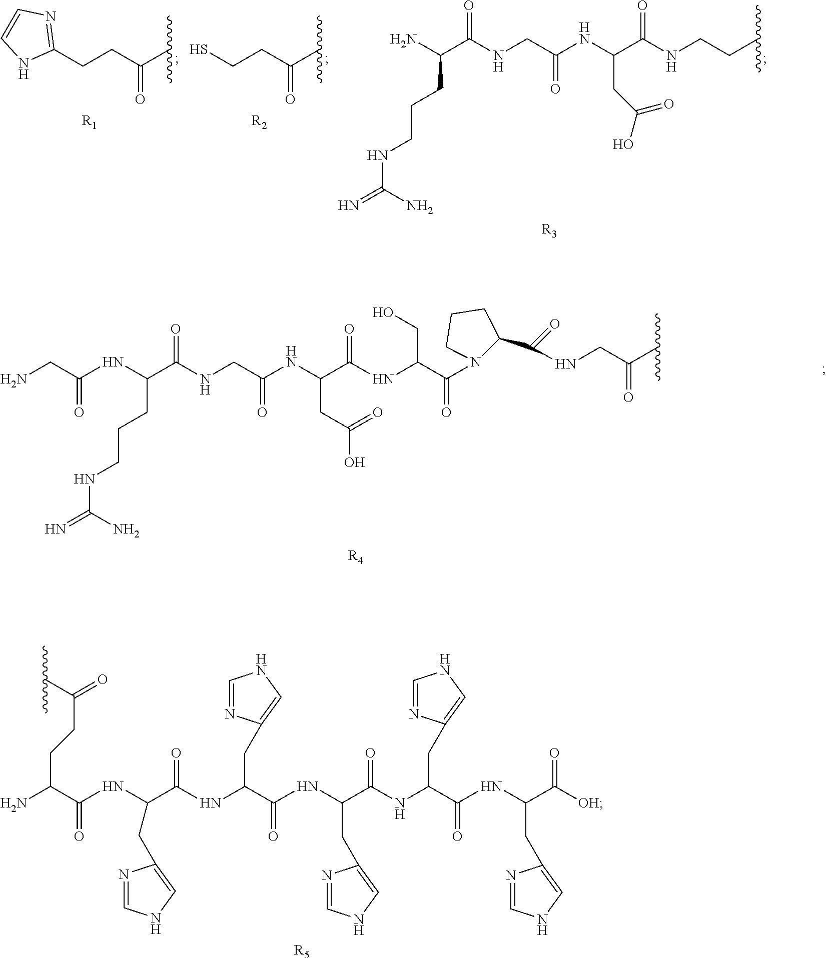

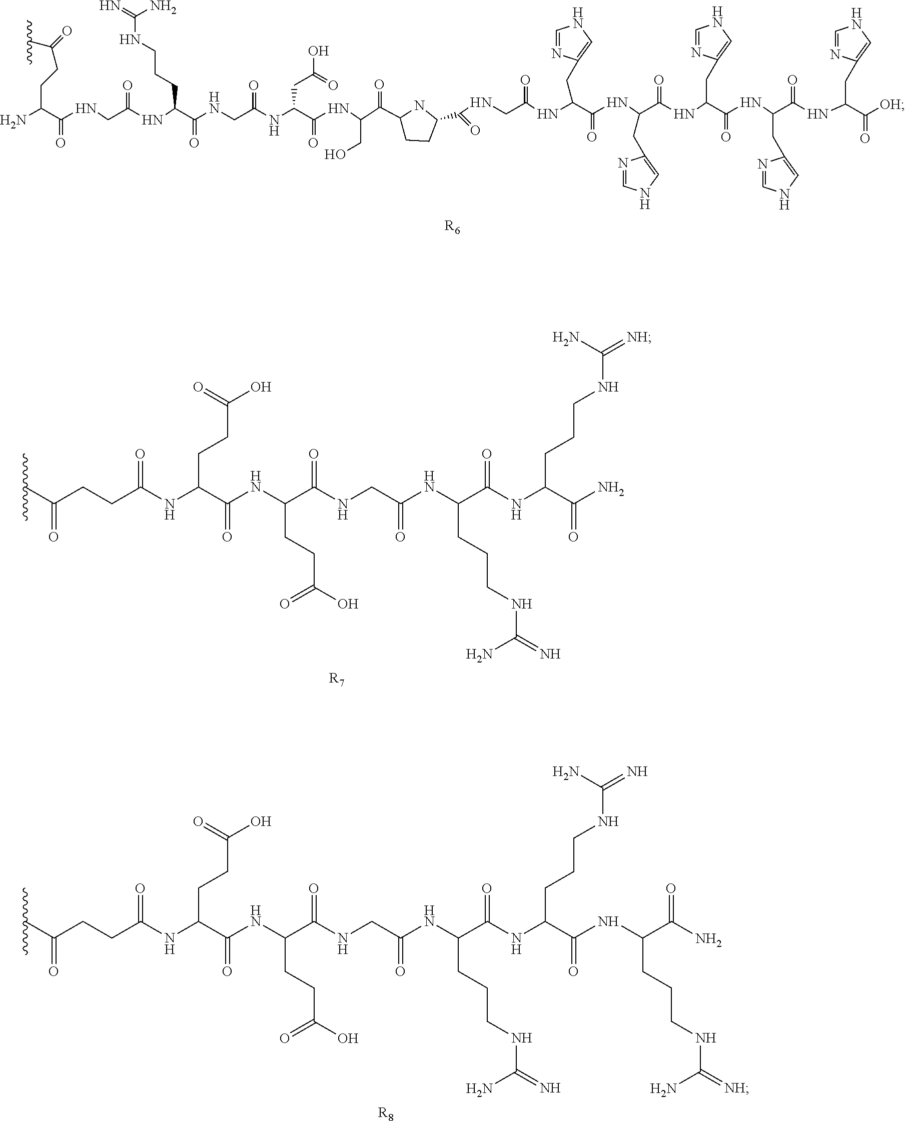

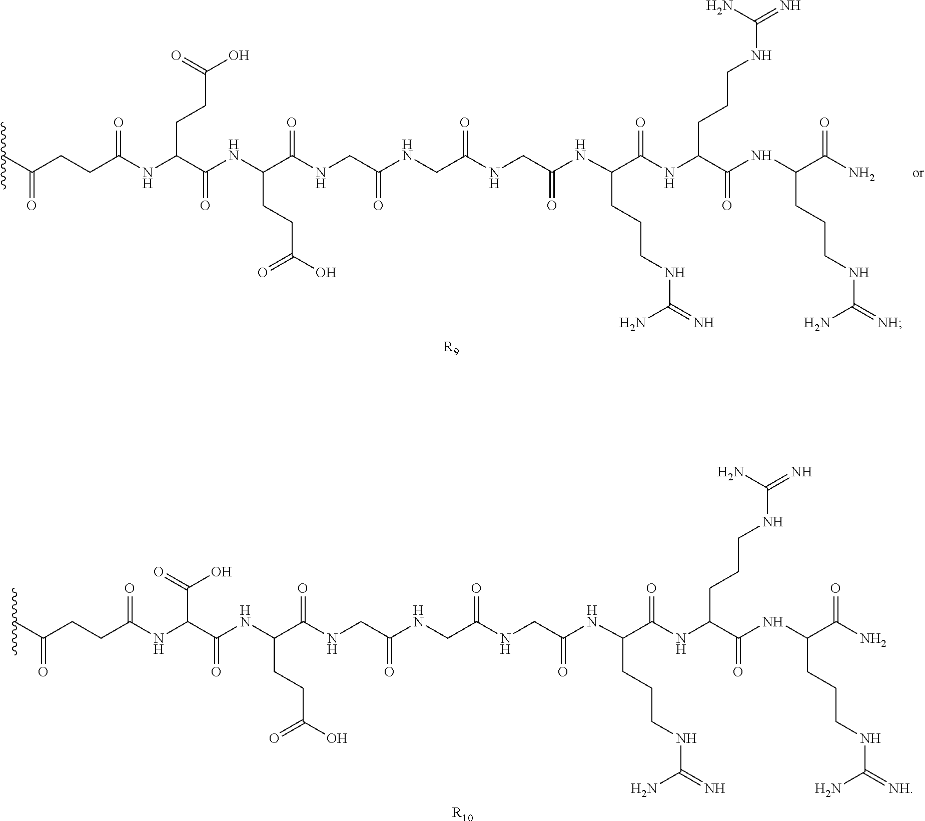

16. The tri-modal nucleic acid delivery composition of claim 1, wherein the helper lipid comprises a neutral helper lipid.

17. The tri-modal nucleic acid delivery composition of claim 1, wherein the helper lipid comprises DOPE (1,2-dioleyl-sn-glycero-3-phosphoethanolamine), DPPC (1,2-dipalmitoyl-sn-glycero-3-phosphocholine), a derivative thereof, or any combination thereof.

18.-19. (canceled)

20. The tri-modal nucleic acid delivery composition of claim 1, having a cationic surfactant/nucleic acid charge ratio (ρ) of ρ≤3.

21. The tri-modal nucleic acid delivery composition of claim 1, having a helper lipid/surfactant molar ratio (r) of r≤10.

22. The tri-modal nucleic acid delivery composition of claim 1, having a molar concentration of peptide enhancer (M_{P}) of M_{P}≤1000 μM, a molar concentration of surfactant (M_{G}) of M_{C}≤46 and a molar concentration of helper lipid (M_{L}) of M_{L}≤300 μM.

23. The tri-modal nucleic acid delivery composition of claim 1, having a surface charge (ζ potential) of −60 mV≤ζ≤60 mV.

24. The tri-modal nucleic acid delivery composition of claim 1, having a particle size of ≥80 nm and ≤350 nm.

25. A kit for delivering a nucleic acid to a cell, the kit comprising a tri-modal nucleic acid delivery composition according to claim 1, and, optionally, instructions for formulating the nucleic acid with the tri-modal nucleic acid delivery composition.

26. A method of delivering a nucleic acid to a cell, said method comprising:
generating a delivery vehicle comprising the nucleic acid by formulating the nucleic acid with the tri-modal nucleic acid delivery composition according to claim 1; and
administering the delivery vehicle to the cell.

27.-28. (canceled)

29. A gemini surfactant comprising two monomeric surfactants linked by a spacer group, the gemini surfactant being covalently functionalized with a functional moiety.

30. (canceled)

31. The gemini surfactant according to claim 29, wherein the gemini surfactant comprises the structure of formula II: 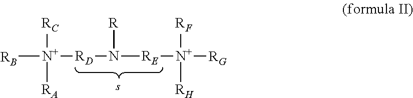
wherein at least one of R_{A}, R_{B}, and R_{C} of a first monomeric surfactant portion comprises an alkyl-based tail having m₁ carbon atoms, and the remaining of R_{A}, R_{B}, and R_{C} are substituents, such as alkyl substituents, which cause the nitrogen to which they are attached to be quaternary;
wherein at least one of R_{F}, R_{G}, and R_{H} of a second monomeric surfactant portion comprises an alkyl-based tail having m₂ carbon atoms, and the remaining of R_{F}, R_{G}, and R_{H} are substituents, such as alkyl substituents, which cause the nitrogen to which they are attached to be quaternary; and
wherein spacer —R_{D}—N(R)—R_{E}— links the first and second monomeric surfactant portions through their respective quaternary nitrogens, R_{D} and R_{E} each represent an alkyl-based group or derivative thereof, R represents the functional moiety and is covalently joined to the nitrogen of the spacer, and s represents the total number of spacer atoms along the shortest linear path running between the quaternary nitrogens of the first and second monomeric surfactant portions.

32. The gemini surfactant according to claim 29, wherein the gemini surfactant comprises the structure of formula III: 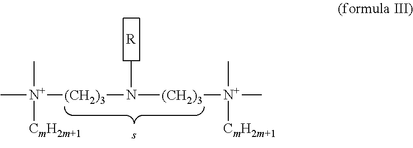
wherein 12≤m≤18, and m may be the same, or different, between the two monomeric surfactant portions;
wherein s is 7; and
wherein R is the functional moiety.

33. (canceled)

34. The gemini surfactant according to claim 29, wherein the functional moiety comprises any one of R₁-R₁₀ as defined in claim 15.

35.-42. (canceled)


SEQUENCE LISTING (16 sequences)

SEQ ID NO: 1 - protein, 7 aa, Artificial Sequence
  PA
GRGDSPG

SEQ ID NO: 2 - protein, 10 aa, Artificial Sequence
  PB
HRRRHRRRHG

SEQ ID NO: 3 - protein, 17 aa, Artificial Sequence
  PC
GRGDSPGHRRRHRRRHG

SEQ ID NO: 4 - protein, 5 aa, Artificial Sequence
  PD
HHHHH

SEQ ID NO: 5 - protein, 12 aa, Artificial Sequence
  PE
GRGDSPGHHHHH

SEQ ID NO: 6 - protein, 15 aa, Artificial Sequence
  PF
HHRHHHHHHHRHHHG

SEQ ID NO: 7 - protein, 22 aa, Artificial Sequence
  PG
GRGDSPGHHRHHHHHHHRHHHG

SEQ ID NO: 8 - protein, 4 aa, Artificial Sequence
  R3
RGDG

SEQ ID NO: 9 - protein, 7 aa, Artificial Sequence
  R4
GRGDSPG

SEQ ID NO: 10 - protein, 13 aa, Artificial Sequence
  R6
EGRGDSPGHHHHH

SEQ ID NO: 11 - protein, 6 aa, Artificial Sequence
  R5
EHHHHH

SEQ ID NO: 12 - protein, 5 aa, Artificial Sequence
  R7 (Suc not shown)
EEGRR

SEQ ID NO: 13 - protein, 6 aa, Artificial Sequence
  R8 (Suc not shown)
EEGRRR

SEQ ID NO: 14 - protein, 8 aa, Artificial Sequence
  R9 (Suc not shown)
EEGGGRRR

SEQ ID NO: 15 - protein, 8 aa, Artificial Sequence
  R10 (Suc not shown)
DEGGGRRR

SEQ ID NO: 16 - protein, 6 aa, Artificial Sequence
  GRGDSP Motif
GRGDSP